SEQUENCE LISTING (22 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 14 aa, Homo sapiens
SKRNIQFSCKNIYR

SEQ ID NO: 3 - protein, 14 aa, Homo sapiens
EKVQTLEAWVIHGG

SEQ ID NO: 4 - protein, 122 aa, synthetic construct [sequence omitted]

SEQ ID NO: 5 - protein, 107 aa, synthetic construct [sequence omitted]

SEQ ID NO: 6 - protein, 5 aa, synthetic construct
  REGION 1..5: note = Antibody HCDR1
SFAMS

SEQ ID NO: 7 - protein, 17 aa, synthetic construct
  REGION 1..17: note = Antibody HCDR2
AISGSGGGTYYADSVKG

SEQ ID NO: 8 - protein, 13 aa, synthetic construct
  REGION 1..13: note = mAb HCDR3
DKILWFGEPVFDY

SEQ ID NO: 9 - protein, 11 aa, synthetic construct
  REGION 1..11: note = Antibody LCDR1
RASQSVSSYLA

SEQ ID NO: 10 - protein, 7 aa, synthetic construct
  REGION 1..7: note = Antibody LCDR2
DASNRAT

SEQ ID NO: 11 - protein, 10 aa, synthetic construct
  REGION 1..10: note = Antibody LCDR3
QQRSNWPPTF

SEQ ID NO: 12 - protein, 452 aa, synthetic construct
  REGION 1..452: note = Antibody HC
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Antibody LC
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 122 aa, synthetic construct
  REGION 1..122: note = mAb 003 VH
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSSAS

SEQ ID NO: 15 - protein, 107 aa, synthetic construct
  REGION 1..107: note = mAb 003 VL
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 16 - protein, 122 aa, synthetic construct
  REGION 1..122: note = mAb 024 Vh
EVQLVQSGAEVKKPGESLKISCKGSGYSFSNYWIGWVRQMPGKGLEWMGIIYPHDSDARYSPSFQGQVTFSADKSISTAYLQWSSLKASDTAMYYCARHVGWGSRYWYFDLWGRGTLVTVSS

SEQ ID NO: 17 - protein, 107 aa, synthetic construct [sequence omitted]

SEQ ID NO: 18 - protein, 120 aa, synthetic construct
  REGION 1..120: note = MOR202VH
QVQLVESGGGLVQPGGSLRLSCAASGFTFSSYYMNWVRQAPGKGLEWVSGISGDPSNTYYADSVKGRFTISRDNSKNTLYLQMNSLRAEDTAVYYCARDLPLVYTGFAYWGQGTLVTVSS

SEQ ID NO: 19 - protein, 109 aa, synthetic construct
  REGION 1..109: note = MOR202VL
DIELTQPPSVSVAPGQTARISCSGDNLRHYYVYWYQQKPGQAPVLVIYGDSKRPSGIPERFSGSNSGNTATLTISGTQAEDEADYYCQTYTGGASLVFGGGTKLTVLGQ

SEQ ID NO: 20 - protein, 120 aa, synthetic construct [sequence omitted]

SEQ ID NO: 21 - protein, 107 aa, synthetic construct [sequence omitted]

SEQ ID NO: 22 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

(19) United States
(12) Patent Application Publication
(10) Pub. No.: US 2026/0139070 A1
(43) Pub. Date: May 21, 2026

(54) Anti-CD38 Antibodies for Treatment of Light Chain Amyloidosis and Other CD38-Positive Hematological Malignancies

(71) Applicants: Janssen Biotech, Inc., Horsham, PA (US); Tufts Medical Center, Inc., Boston, MA (US)

(72) Inventors: Chakra Chaulagain, Broward, FL (US); Raymond Comenzo, Boston, MA (US); Parul Doshi, Chester Springs, PA (US); Xun Ma, Boston, MA (US); Amy Sasser, Doylestown, PA (US)

(21) Appl. No.: 19/449,026

(22) Filed: Jan. 14, 2026

Related U.S. Application Data
(63) Continuation of application No. 18/774,480, filed on Jul. 16, 2024.
Application No. 18/774,480 is a continuation of application No. 16/986,214, filed on Aug. 5, 2020, now Pat. No. 12,091,466.
Application No. 16/986,214 is a continuation of application No. 15/160,476, filed on May 20, 2016, now Pat. No. 10,766,965.
(60) Provisional application No. 62/164,206, filed on May 20, 2015.
(60) Provisional application No. 62/214,586, filed on Sep. 4, 2015.

(51) Int. Cl. C07K 16/28; A61K 9/00; A61K 31/573; A61K 31/664; A61K 31/69; A61K 38/05; A61K 38/47; A61K 39/00; A61K 39/395; A61K 45/06

(52) U.S. Cl. CPC C07K 16/2896; A61K 9/0019; A61K 31/573; A61K 31/664; A61K 31/69; A61K 38/05; A61K 38/47; A61K 39/3955; A61K 45/06; C12Y 302/01035; A61K 2039/505; C07K 2317/21; C07K 2317/24; C07K 2317/56; C07K 2317/565; C07K 2317/732

(57) ABSTRACT

The present invention relates to methods of treatment of light chain amyloidosis and other CD38-positive hematological malignancies with anti-CD38 antibodies.

### RELATED APPLICATIONS

This application is a continuation of U.S. application Ser. No. 18/774,480, filed on Jul. 16, 2024, which is a continuation of U.S. application Ser. No. 16/986,214, filed on Aug. 5, 2020, now U.S. Pat. No. 12,091,466, issued on Sep. 17, 2024, which is a continuation of U.S. application Ser. No. 15/160,476, filed on May 20, 2016, now U.S. Pat. No. 10,766,965, issued on Sep. 8, 2020, which claims the benefit of U.S. Provisional Application No. 62/164,206, filed on May 20, 2015 and claims the benefit of U.S. Provisional Application No. 62/214,586, filed on Sep. 4, 2015. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 01482017072.xml; created Jan. 13, 2026, 29,348 Bytes in size.

### FIELD OF THE INVENTION

The present invention relates to methods of treatment of light chain amyloidosis and other CD38-positive hematological malignancies.

### BACKGROUND

B-cell malignancies include B-cell chronic lymphocytic leukemia, mantle cell lymphoma, Burkitt lymphoma, follicular lymphoma, diffuse large B-cell lymphoma, multiple myeloma, Hodgkin's lymphoma, hairy cell leukemia, primary effusion lymphoma and AIDS-related Non-Hodgkin's Lymphoma. B-cell malignancies comprise more than 85% of diagnosed lymphomas.

Multiple myeloma (MM) is characterized by the latent accumulation of secretory plasma cells in bone marrow with a low proliferative index and an extended life span. The disease ultimately attacks bones and bone marrow, resulting in multiple tumors and lesions throughout the skeletal system. Approximately 1% of all cancers and slightly more than 10% of all hematologic malignancies can be attributed to multiple myeloma. Incidence of multiple myeloma increases in the aging population, with the median age at time of diagnosis being about 61 years.

Light chain amyloidosis (AL) (also called systemic amyloidosis) is a clonal plasma cell disorder in which fragments of misfolded immunoglobulin light chains are deposited in tissues. Monoclonal plasma cells in the bone marrow produce the misfolded immunoglobulin light chains that accumulate in tissues and cause toxicity in vital organs leading to organ failure and death (Comenzo et al., Leukemia 26:2317-25, 2012). The clinical features depend on the organs involved; amyloidosis frequently manifests in kidneys, heart, skin, nervous system and in soft tissues, such as the tongue (Merlini and Belotti, NEJM, 349:583-596, 2003), resulting in albuminuria and renal failure, heart failure, arrhythmias, risk of sudden cardiac death, hepatomegaly, bloating, early satiety, paresthesias, dysthesias, orthostatic hypotension, constipation, or diarrhea (Chaulagain and Comenzo; Curr Hematol Malig Rep 8:291-8, 2013).

CD38 is a type II membrane protein having function in receptor-mediated adhesion and signaling as well as mediating calcium mobilization via its ecto-enzymatic activity, catalyzing formation of cyclic ADP-ribose (cADPR) from NAD⁺ and also hydrolyzing cADPR into ADP-ribose (ADPR). CD38 mediates cytokine secretion and activation and proliferation of lymphocytes (Funaro et al., J Immunology 145:2390-6, 1990; Guse et al., Nature 398:70-3, 1999), and via its NAD glycohydrolase activity regulates extracellular NAD⁺ levels which have been implicated in modulating the regulatory T-cell compartment (Adriouch et al., 14:1284-92, 2012; Chiarugi et al., Nature Reviews 12:741-52, 2012).

CD38 is expressed on multiple myeloma malignant plasma cells, and is implicated in various hematological malignancies.

Current treatments for light chain amyloidosis and multiple myeloma include various chemotherapeutic agents with or without autologous stem cell transplantation. However, both diseases remain largely incurable. Thus, there is a need for additional therapeutics for multiple myeloma and light chain amyloidosis.

### SUMMARY

The invention provides for a method of treating a patient having a CD38-positive hematological malignancy, comprising administering to the patient in need thereof an anti-CD38 antibody for a time sufficient to treat the CD38-positive hematological malignancy, wherein the patient is undergoing hematopoietic stem cell transplantation (HSCT).

The invention also provides for a method of treating a patient having light chain amyloidosis (AL), comprising administering to the patient in need thereof an anti-CD38 antibody for a time sufficient to treat AL.

The invention also provides for a method of treating a patient having light chain amyloidosis (AL), comprising administering to the patient in need thereof an anti-CD38 antibody for a time sufficient to treat AL, wherein the patient is undergoing hematopoietic stem cell transplantation (HSCT).

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 shows the relative expression of CD38, CD32B, IL6R, gp130 and CD16 in transcriptional profiles of CD138⁺ clonal plasma cells of newly diagnosed patients with AL (n=16 AL patients, GEO GSE24128). LnQLE: natural log of the quantitative level of expression.

FIG. 2 shows the percentage (%) of NK cells of the mononuclear cells in the peripheral blood of AL patients at 3 weeks after stem cell transplant (SCT).

FIG. 3 shows that CD38 is expressed on CD34⁺ hematopoietic progenitor cells.

FIG. 4A shows that DARZALEX™ (daratumumab) has no effect on proliferation of thawed cryopreserved unselected mobilized blood progenitor cells, assessed by the ability of the progenitor cells to form similar numbers of colonies in the presence of 500 ng or 1000 ng/ml DARZALEX™ (daratumumab) when compared to isotype control. In this assay, colony forming unit granulocyte-macrophage (CFU-GM) was assessed after 14 days of culture in methylcellulose in the presence of DARZALEX™ (daratumumab), isotype control or no antibody control, and % colonies formed was plotted as a function of no antibody control. *p<0.05, paired t test. All assays were done in triplicates and 3 independent experiments were performed with progenitor cell specimens from 3 different patients. Data shows mean+/−SD.

FIG. 4B shows that DARZALEX™ (daratumumab) has no effect on proliferation of thawed cryopreserved unselected mobilized blood progenitor cells, assessed by the ability of the progenitor cells to form similar numbers colonies in the presence of 500 ng or 1000 ng/ml DARZALEX™ (daratumumab) when compared to the isotype control. In this assay, blast forming unit erythroid (BFU-E) was assessed after 14 day growth in methylcellulose in the presence of DARZALEX™ (daratumumab), isotype control or no antibody control, and % colonies formed was plotted as a function of no antibody control. All assays were done in triplicates and 3 independent experiments were performed with progenitor cell specimens from 3 different patients. Data shows mean+/−SD.

FIG. 5A shows that DARZALEX™ (daratumumab) has no effect on proliferation of fresh unselected mobilized blood progenitor cells assessed by their ability to form similar numbers of colonies in methylcellulose in the presence of 500 ng or 1000 ng/ml DARZALEX™ (daratumumab) when compared to isotype control. Colony formation was measured at day 14 as CFU-GM and % colonies formed was plotted as a function of no antibody control. All assays were done in triplicates and 3 independent experiments were performed with progenitor cell specimens from 3 different patients. Data shows mean+/−SD.

FIG. 5B shows that DARZALEX™ (daratumumab) has no effect on proliferation of fresh unselected mobilized blood progenitor cells assessed by their ability to form similar numbers of colonies in methylcellulose in the presence of 500 ng or 1000 ng/ml DARZALEX™ (daratumumab) when compared to isotype control. Colony formation was measured at day 14 as BFU-E and % colonies formed was plotted as a function of no antibody control. All assays were done in triplicates and 3 independent experiments were performed with progenitor cell specimens from 3 different patients. Data shows mean+/−SD.

FIG. 6A shows that DARZALEX™ (daratumumab) does not kill CD34-selected hematopoietic progenitor cells by CDC despite high CD38 expression on the cells. CD34 cells were incubated 1 hour with serum with high complement with no antibody (CTL), 500 ng/ml DARZALEX™ (daratumumab) (Dara) or 500 ng/ml isotype control (Iso), after which cells were plated on semisolid media. Colony formation was measured at day 14 as CFU-GM per 500 CD34-selected cells. The results are from triplicate assays with cells from 1 patient with MM and 2 patients with AL.

FIG. 6B shows that DARZALEX™ (daratumumab) does not kill CD34-selected hematopoietic progenitor cells by CDC despite high CD38 expression on the cells. CD34 cells were incubated 1 hour with serum with high complement, no antibody (CTL), 500 ng/ml DARZALEX™ (daratumumab) (Dara) or 500 ng/ml isotype control (Iso), after which cells were plated on semisolid media. Colony formation was measured at day 14 as BFU-E per 500 CD34-selected cells. There were more BFU-E in plates with DARZALEX™ (daratumumab) that reached statistical significance. The results are from triplicate assays with cells from 1 patient with MM and 2 patients with AL. (* p<0.01).

FIG. 7A shows that DARZALEX™ (daratumumab) has no adverse effect on fresh CD34-selected granulcytic-monocytic progenitor cells. Isolated CD34⁺ cells were incubated in medium containing no antibody (CTL; control), DARZALEX™ (daratumumab) (Dara) or isotype control (Iso) at 500 ng/ml or 1000 ng/ml as indicated. Cells were placed in methylcellulose and colony formation was measured as CFU-GM per 500 CD34-selected cells at day 14. The results are from triplicate assays with cells from 1 patient with MM and 2 patients with AL.

FIG. 7B shows that DARZALEX™ (daratumumab) has no adverse effect on fresh CD34-selected erythroid progenitor cells. Isolated CD34-cells were incubated in medium containing no antibody (CTL; control), daratumumab or isotype control (Iso) at 500 ng/ml or 1000 ng/ml. Cells were placed in methylcellulose and colony formation was measured as BFU-E per 500 CD34-selected cells at day 14. The results are from triplicate assays with cells from 2 patients with MM and 1 patient with AL. * p<0.02.

FIG. 8 shows that DARZALEX™ (daratumumab)-mediated ADCC was influenced by FCγRIIIa-158aa polymorphisms. Patients with 158V/V (V/V) and 158F/V (F/V) genotypes demonstrated an increased response rate when compared to the patients with 158F/F (F/F) genotype (vertical line is median for each group, P<0.05, Mann Whitney, two-tailed).

### DETAILED DESCRIPTION

A description of example embodiments follows.

“CD38” refers to the human CD38 protein (synonyms: ADP-ribosyl cyclase 1, cADPr hydrolase 1, cyclic ADP-ribose hydrolase 1). Human CD38 has an amino acid sequence shown in GenBank accession number NP_001766 and in SEQ ID NO: 1. it is well known that CD38 is a single pass type II membrane protein with amino acid residues 1-21 representing the cytosolic domain, amino acid residues 22-42 representing the transmembrane domain, and residues 43-300 representing the extracellular domain of CD38.

SEQ ID NO: 1
MANCEFSPVSGDKPCCRLSRRAQLCLGVSILVLILVVVLAVVVPRWRQQWS
GPGTTKRFPETVLARCVKYTEIHPEMRHVDCQSVWDAFKGAFISKHPCNIT
EEDYQPLMKLGTQTVPCNKILLWSRIKDLAHQFTQVQRDMFTLEDTLLGYL
ADDLTWCGEFNTSKINYQSCPDWRKDCSNNPVSVFWKTVSRRFAEAACDVV
HVMLNGSRSKIFDKNSTFGSVEVHNLQPEKVQTLEAWVIHGGREDSRDLCQ
DPTIKELESIISKRNIQFSCKNIYRPDKFLQCVKNPEDSSCTSEI

“Antibodies” as used herein is meant in a broad sense and includes immunoglobulin molecules including monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄. Antibody light chains of any vertebrate species can be assigned to one of two clearly distinct types, namely kappa (κ) and lambda (λ), based on the amino acid sequences of their constant domains.

“Antibody fragments” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (VH), or a light chain variable region (VL). Antibody fragments include a Fab fragment, a monovalent fragment consisting of the VL, VH, CL and CHI domains; a F(ab)₂ fragment, a bivalent fragment comprising two Fab fragments linked by a disulfide bridge at the hinge region; a Fd fragment consisting of the VH and CHI domains; a Fv fragment consisting of the VL and VH domains of a single arm of an antibody; a domain antibody (dAb) fragment (Ward et al., Nature 341:544-6, 1989), which consists of a VH domain. VH and VL domains can be engineered and linked together via a synthetic linker to form various types of single chain antibody designs in which the VH/VL domains pair intramolecularly, or intermolecularly in those cases in which the VH and VL domains are expressed by separate single chain antibody constructs, to form a monovalent antigen binding site, such as single chain Fv (scFv) or diabody; described for example in Intl. Pat. Publ. Nos. WO1998/44001, WO1988/01649, WO1994/13804, and WO1992/01047. These antibody fragments are obtained using known techniques known to those of skill in the art, and the fragments are screened for utility in the same manner as are full length antibodies.

“Isolated antibody” refers to an antibody or antibody fragment that is substantially free of other antibodies having different antigenic specificities (e.g., an isolated antibody specifically binding CD38 is substantially free of antibodies that specifically bind antigens other than human CD38). An isolated antibody that specifically binds CD38, however, can have cross-reactivity to other antigens, such as orthologs of human CD38, such as Macaca fascicularis (cynomolgus) CD38. Moreover, an isolated antibody may be substantially free of other cellular material and/or chemicals.

An antibody variable region consists of a “framework” region interrupted by three “antigen binding sites”. The antigen binding sites are defined using various terms: Complementarity Determining Regions (CDRs), three in the VH (HCDR1, HCDR2, HCDR3) and three in the VL (LCDR1, LCDR2, LCDR3) are based on sequence variability (Wu and Kabat J Exp Med 132:211-50, 1970; Kabat et al Sequences of Proteins of Immunological Interest, 5th Ed. Public Health Service, National Institutes of Health, Bethesda, Md., 1991); “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro, Mol Recognit 17:132-43, 2004). The International ImMunoGeneTics (IMGT) database (www_imgt_org) provides a standardized numbering and definition of antigen-binding sites. The correspondence between CDRs, HVs and IMGT delineations is described in Lefranc et al., Dev Comparat Immunol 27:55-77, 2003.

“Chothia residues” as used herein are the antibody VL and VH residues numbered according to Al-Lazikani (Al-Lazikani et al., J Mol Biol 273:927-48, 1997).

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human-adapted” antibodies or “human framework adapted (HFA)” antibodies refers to humanized antibodies adapted according to methods described in U.S. Pat. Publ. No. US2009/0118127. Human-adapted antibodies are humanized by selecting the acceptor human frameworks based on the maximum CDR and FR similarities, length compatibilities and sequence similarities of CDR1 and CDR2 loops and a portion of light chain CDR3 loops.

“Human antibody” refers to an antibody having heavy and light chain variable regions in which both the framework and the antigen binding sites are derived from sequences of human origin. If the antibody contains a constant region, the constant region also is derived from sequences of human origin.

A human antibody comprises heavy and/or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice or rat carrying human immunoglobulin loci as described herein. A “human antibody” may contain amino acid differences when compared to the human germline or rearranged immunoglobulin sequences due to for example naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, “human antibody” may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, for example as described in Shi et al., J Mol Biol 397:385-96, 2010 and Intl. Pat. Publ. No. WO2009/085462). Antibodies in which antigen binding sites are derived from a non-human species are not included in the definition of human antibody.

Isolated humanized antibodies may be synthetic. Human antibodies, while derived from human immunoglobulin sequences, may be generated using systems such as phage display incorporating synthetic CDRs and/or synthetic frameworks, or can be subjected to in vitro mutagenesis to improve antibody properties, resulting in antibodies that do not naturally exist within the human antibody germline repertoire in vivo.

“Recombinant antibody” includes all antibodies that are prepared, expressed, created or isolated by recombinant means, such as antibodies isolated from an animal (for example a mouse or a rat) that is transgenic or transchromosomal for human immunoglobulin genes or a hybridoma prepared therefrom, antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange such as bispecific antibodies.

“Monoclonal antibody” refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes. “Monoclonal antibody” therefore refers to an antibody population with single amino acid composition in each heavy and each light chain, except for possible well known alterations such as removal of C-terminal lysine from the antibody heavy chain. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” refers to a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or noncontiguous amino acids that form a conformational spatial unit. For a noncontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions or deletions.

“In combination with” means that two or more therapeutics can be administered to a subject together in a mixture, concurrently as single agents or sequentially as single agents in any order.

“Treat” or “treatment” refers to therapeutic treatment wherein the object is to slow down (lessen) an undesired physiological change or disease, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those subjects already with the undesired physiological change or diseases well as those subjects prone to have the physiological change or disease.

“Inhibits growth” (e.g. referring to cells, such as tumor cells) refers to a measurable decrease in the cell growth in vitro or in vivo when the cell is contacted with a therapeutic or a combination of therapeutics or drugs, when compared to the growth of the same cell grown in appropriate control conditions well known to the skilled in the art. Inhibition of growth of a cell in vitro or in vivo may be at least about 10%, 20%, 30%, 40%, 50%, 60%, 70%, 80%, 90%, 99%, or 100%. Inhibition of cell growth can occur by a variety of mechanisms, for example by antibody-dependent cell-mediated cytotoxicity (ADCC), antibody-dependent cellular phagocytosis (ADCP), complement dependent cytotoxicity (CDC), apoptosis, necrosis, or by inhibition of cell proliferation.

“Therapeutically effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective amount of a therapeutic or combination of therapeutics include, for example, improved well-being of the patient, reduction of a tumor burden, arrested or slowed growth of a tumor, and/or absence of metastasis of cancer cells to other locations in the body.

“Patient” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. The terms “patient” and “subject” are used interchangeably herein.

The invention provides for a method of treating patients having a CD38-positive hematological malignancy undergoing hematopoietic stem cell transplantation with an anti-CD38 antibody that does not kill (e.g. mediate killing of) CD34 hematopoietic stem cells within the transplant, which cells are also CD38 positive. The invention also provides for a method for treating patients having light chain amyloidosis. The invention is based, at least in part, on the discovery that an anti-CD38 antibody DARZALEX™ (daratumumab) is effective in killing AL plasma cells, but does not kill CD38 CD34 hematopoietic stem cells isolated from patients having light chain amyloidosis or multiple myeloma, allowing combination treatment with DARZALEX™ (daratumumab) and hematopoietic stem cell transplantation.

The methods of the invention may be used to treat an animal patient belonging to any classification. Examples of such animals include mammals such as humans, rodents, dogs, cats and farm animals.

The invention provides for a method of treating a patient having a CD38-positive hematological malignancy, comprising administering to the patient in need thereof an anti-CD38 antibody for a time sufficient to treat the CD38-positive hematological malignancy, wherein the patient is undergoing hematopoietic stem cell transplantation (HSCT).

“CD38-positive hematological malignancy” refers to a hematological malignancy characterized by the presence of tumor cells expressing CD38 including leukemias, lymphomas, myeloma and plasma cell disorders. Examples of CD38-positive hematological malignancies include precursor B-cell lymphoblastic leukemia/lymphoma and B-cell non-Hodgkin's lymphoma, acute promyelocytic leukemia, acute lymphoblastic leukemia and mature B-cell neoplasms, such as B-cell chronic lymphocytic leukemia (CLL)/small lymphocytic lymphoma (SLL), B-cell acute lymphocytic leukemia, B-cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B-cell lymphoma (MALT type, nodal and splenic type), hairy cell leukemia, diffuse large B-cell lymphoma (DLBCL), Burkitt's lymphoma (BL), plasmacytoma, multiple myeloma (MM), plasma cell leukemia, post-transplant lymphoproliferative disorder, Waldenstrom's macroglobulinemia, plasma cell leukemias, anaplastic large-cell lymphoma (ALCL) and light chain amyloidosis (AL).

“Plasma cell disorder” as used herein refers to disorders characterized by clonal plasma cells, and includes multiple myeloma, light chain amyloidosis and Waldenstrom's macroglobulinemia. Light chain amyloidosis and Waldenstrom's macrogloblinemia can arise independently from multiple myeloma. They may also present simultaneously with multiple myeloma, and develop either before or after the development of multiple myeloma.

The definitions “CD-38 positive hematological malignancy” and “plasma cell disorders” may thus be partially overlapping.

In some embodiments, the CD38-positive hematological malignancy is light chain amyloidosis (AL).

In some embodiments, the CD38-positive hematological malignancy is multiple myeloma (MM).

In some embodiments, the CD38-positive hematological malignancy is Waldenstrom's macroglobulinemia.

In some embodiments, the CD38-positive hematological malignancy is diffuse large B-cell lymphoma (DLBCL).

In some embodiments, the CD38-positive hematological malignancy is non-Hodgkin's lymphoma.

In some embodiments, the CD38-positive hematological malignancy is acute lymphoblastic leukemia (ALL).

In some embodiments, the CD38-positive hematological malignancy is follicular lymphoma (FL).

In some embodiments, the CD38-positive hematological malignancy is Burkitt's lymphoma (BL).

In some embodiments, the CD38-positive hematological malignancy is mantle cell lymphoma (MCL).

In some embodiments, the CD38-positive hematological malignancy is a plasma cell disorder.

In some embodiments, the CD38-positive hematological malignancy is light chain amyloidosis (AL), multiple myeloma (MM), acute lymphoblastic leukemia (ALL), non-Hodgkin's lymphoma, diffuse large B-cell lymphoma (DLBCL), Burkitt's lymphoma (BL), follicular lymphoma (FL) or mantle-cell lymphoma (MCL).

Exemplary B-cell non-Hodgkin's lymphomas are lymphomatoid granulomatosis, primary effusion lymphoma, intravascular large B-cell lymphoma, mediastinal large B-cell lymphoma, heavy chain diseases (including γ, μ, and a disease), lymphomas induced by therapy with immunosuppressive agents, such as cyclosporine-induced lymphoma, and methotrexate-induced lymphoma.

In some embodiments, the disorder involving cells expressing CD38 is Hodgkin's lymphoma.

Other examples of disorders involving cells expressing CD38 include malignancies derived from T and NK cells including mature T cell and NK cell neoplasms including T-cell prolymphocytic leukemia, T-cell large granular lymphocytic leukemia, aggressive NK cell leukemia, adult T-cell leukemia/lymphoma, extranodal NK/T cell lymphoma, nasal type, 78 enteropathy-type T-cell lymphoma, hepatosplenic T-cell lymphoma, subcutaneous panniculitis-like T-cell lymphoma, blastic NK cell lymphoma, Mycosis Fungoides/Sezary Syndrome, primary cutaneous CD30 positive T-cell lymphoproliferative disorders (primary cutaneous anaplastic large cell lymphoma C-ALCL, lymphomatoid papulosis, borderline lesions), angioimmunoblastic T-cell lymphoma, peripheral T-cell lymphoma unspecified, and anaplastic large cell lymphoma.

Examples of malignancies derived from myeloid cells include acute myeloid leukemia, including acute promyelocytic leukemia, and chronic myeloproliferative diseases, including chronic myeloid leukemia.

The invention also provides for a method of treating a patient having light chain amyloidosis (AL), comprising administering to the patient in need thereof an anti-CD38 antibody for a time sufficient to treat AL

The invention also provides for a method of treating a patient having light chain amyloidosis, comprising administering to the patient in need thereof an anti-CD38 antibody for a time sufficient to treat the light chain amyloidosis, wherein the patient is undergoing hematopoietic stem cell transplantation (HSCT).

The invention also provides for a method of treating a patient having multiple myeloma, comprising administering to the patient in need thereof an anti-CD38 antibody for a time sufficient to treat the multiple myeloma, wherein the patient is undergoing hematopoietic stem cell transplantation (HSCT).

In some embodiments, the anti-CD38 antibody competes for binding to CD38 with an antibody comprising a heavy chain variable region (VH) of SEQ ID NO: 4 and a light chain variable region (VL) of SEQ ID NO: 5.

In some embodiments, the anti-CD38 antibody binds at least to the region SKRNIQFSCKNIYR (SEQ ID NO: 2) and the region EKVQTLEAWVIHGG (SEQ ID NO: 3) of human CD38 (SEQ ID NO: 1).

In some embodiments, the anti-CD38 antibody comprises a heavy chain complementarity determining region (HCDR) 1, a HCDR2 and a HCDR3 of SEQ ID NOs: 6, 7 and 8, respectively.

In some embodiments, the anti-CD38 antibody comprises a light chain complementarity determining region (LCDR) 1, a LCDR2 and a LCDR3 of SEQ ID NOs: 9, 10 and 11, respectively.

In some embodiments, the anti-CD38 antibody comprises the HCDR1, the HCDR2 and the HCDR3 of SEQ ID NOs: 6, 7 and 8, respectively, and the LCDR1, the LCDR2 and the LCDR3 of SEQ ID NOs: 9, 10 and 11, respectively.

In some embodiments, the anti-CD38 antibody comprises a heavy chain variable region (VH) amino acid sequence that is 95%, 96%, 97%, 98%, 99% or 100% identical to that of SEQ ID NO: 4 and a light chain variable region (VL) amino acid sequence that is 95%, 96%, 97%, 98%, 99% or 100% identical to that of SEQ ID NO: 5.

In some embodiments, the anti-CD38 antibody comprises the VH of SEQ ID NO: 4 and the VL of SEQ ID NO: 5.

In some embodiments, the anti-CD38 antibody comprises a heavy chain comprising an amino acid sequence that is 95%, 96%, 97%, 98%, 99% or 100% identical to that of SEQ ID NO: 12 and a light chain comprising an amino acid sequence that is 95%, 96%, 97%, 98%, 99% or 100% identical to that of SEQ ID NO: 13.

In some embodiments, the anti-CD38 antibody comprises the heavy chain of SEQ ID NO: 12 and the light chain of SEQ ID NO: 13.

The epitope of the antibody includes some or all of the residues having the sequences shown in SEQ ID NO: 2 or SEQ ID NO: 3. In some embodiments, the antibody epitope comprises at least one amino acid in the region SKRNIQFSCKNIYR (SEQ ID NO: 2) and at least one amino acid in the region EKVQTLEAWVIHGG (SEQ ID NO: 3) of human CD38 (SEQ ID NO: 1). In some embodiments, the antibody epitope comprises at least two amino acids in the region SKRNIQFSCKNIYR (SEQ ID NO: 2) and at least two amino acids in the region EKVQTLEAWVIHGG (SEQ ID NO: 3) of human CD38 (SEQ ID NO: 1). In some embodiments, the antibody epitope comprises at least three amino acids in the region SKRNIQFSCKNIYR (SEQ ID NO: 2) and at least three amino acids in the region EKVQTLEAWVIHGG (SEQ ID NO: 3) of human CD38 (SEQ ID NO: 1).

An exemplary antibody that binds to the region SKRNIQFSCKNIYR (SEQ ID NO: 2) and the region EKVQTLEAWVIHGG (SEQ ID NO: 3) of human CD38 (SEQ ID NO: 1) is DARZALEX™ (daratumumab).

An exemplary anti-CD38 antibody that may be used in the methods of the invention is DARZALEX™ (daratumumab). DARZALEX™ (daratumumab) comprises the heavy chain variable region (VH) and the light chain variable region (VL) amino acid sequences shown in SEQ ID NO: 4 and 5, respectively, heavy chain CDRs HCDR1, HCDR2 and HCDR3 of SEQ ID NOs: 6, 7 and 8, respectively, and light chain CDRs LCDR1, LCDR2 and LCDR3 of SEQ ID NOs: 9, 10 and 11, respectively, and is of IgG1/κ subtype and described in U.S. Pat. No. 7,829,693. DARZALEX™ (daratumumab) heavy chain amino acid sequence is shown in SEQ ID NO: 12 and light chain amino acid sequence shown in SEQ ID NO: 13.

Antibodies may be evaluated for their competition with a reference antibody, for example DARZALEX™ (DARZALEX™ (daratumumab)) having the VH of SEQ ID NO: 4 and the VL of SEQ ID NO: 5 for binding to CD38 using well known in vitro methods. In an exemplary method, CHO cells recombinantly expressing CD38 may be incubated with an unlabeled reference antibody for 15 min at 4° C., followed by incubation with an excess of a fluorescently labeled test antibody for 45 min at 4° C. After washing in PBS/BSA, fluorescence may be measured by flow cytometry using standard methods. In another exemplary method, the extracellular domain of CD38 may be coated on the surface of an ELISA plate. Excess of an unlabeled reference antibody may be added for about 15 minutes and subsequently a biotinylated test antibody may be added. After washes in PBS/Tween, binding of the biotinylated test antibody may be detected using horseradish peroxidase (HRP)-conjugated streptavidine and the signal detected using standard methods. It is readily apparent that in the competition assays, the reference antibody may be labelled and the test antibody unlabeled. The test antibody competes with the reference antibody when the reference antibody inhibits binding of the test antibody, or the test antibody inhibits binding of the reference antibody to CD38 by at least 80%, for example 81%, 82%, 83%, 84%, 85%, 86%, 87%, 88%, 89%, 90%, 91%, 92%, 93%, 94%, 95%, 96%, 97%, 98%, 99% or 100%. The epitope of the test antibody may further be defined for example by peptide mapping or hydrogen/deuterium protection assays using known methods, or by crystal structure determination.

Antibodies binding to the region SKRNIQFSCKNIYR (SEQ ID NO: 2) and the region EKVQTLEAWVIHGG (SEQ ID NO: 3) of human CD38 (SEQ ID NO: 1).may be generated for example by immunizing mice with peptides having the amino acid sequences shown in SEQ ID NOs: 2 and 3 using standard methods and as described herein, and characterizing the obtained antibodies for binding to the peptides using for example ELISA or mutagenesis studies.

SEQ ID NO: 2
SKRNIQFSCKNIYR
SEQ ID NO: 3
EKVQTLEAWVIHGG
SEQ ID NO: 4
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSS
SEQ ID NO: 5
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK
SEQ ID NO: 6
SFAMS
SEQ ID NO: 7
AISGSGGGTYYADSVKG
SEQ ID NO: 8
DKILWFGEPVFDY
SEQ ID NO: 9
RASQSVSSYLA
SEQ ID NO: 10
DASNRAT
SEQ ID NO: 11
QQRSNWPPTF
SEQ ID NO: 12
EVQLLESGGGLVQPGGSLRLSCAVSGFTFNSFAMSWVRQAPGKGLEWVSA
ISGSGGGTYYADSVKGRFTISRDNSKNTLYLQMNSLRAEDTAVYFCAKDK
ILWFGEPVFDYWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK
SEQ ID NO: 13
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Other exempary anti-CD38 antibodies that may be used in the methods of the invention are:

mAb003 comprising the VH and VL sequences of SEQ ID NOs: 14 and 15, respectively and described in U.S. Pat. No. 7,829,693. The VH and the VL of mAb003 may be expressed as IgG1/κ.

SEQ ID NO: 14
QVQLVQSGAEVKKPGSSVKVSCKASGGTFSSYAFSWVRQAPGQGLEWMGR
VIPFLGIANSAQKFQGRVTITADKSTSTAYMDLSSLRSEDTAVYYCARDD
IAALGPFDYWGQGTLVTVSSAS
SEQ ID NO: 15
DIQMTQSPSSLSASVGDRVTITCRASQGISSWLAWYQQKPEKAPKSLIYA
ASSLQSGVPSRFSGSGSGTDFTLTISSLQPEDFATYYCQQYNSYPRTFGQ
GTKVEIK;

mAb024 comprising the VH and VL sequences of SEQ ID NOs: 16 and 17, respectively, described in U.S. Pat. No. 7,829,693. The VH and the VL of mAb024 may be expressed as IgG1/κ.

SEQ ID NO: 16
EVQLVQSGAEVKKPGESLKISCKGSGYSFSNYWIGWVRQMPGKGLEWMGI
IYPHDSDARYSPSFQGQVTFSADKSISTAYLQWSSLKASDTAMYYCARHV
GWGSRYWYFDLWGRGTLVTVSS
SEQ ID NO: 17
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPTFGQ
GTKVEIK;

MOR-202 (MOR-03087) comprising the VH and VL sequences of SEQ ID NOs: 18 and 19, respectively, described in U.S. Pat. No. 8,088,896. The VH and the VL of MOR-202 may be expressed as IgG1/κ.

SEQ ID NO: 18
QVQLVESGGGLVQPGGSLRLSCAASGFTFSSYYMNWVRQAPGKGLEWVSG
ISGDPSNTYYADSVKGRFTISRDNSKNTLYLQMNSLRAEDTAVYYCARDL
PLVYTGFAYWGQGTLVTVSS
SEQ ID NO: 19
DIELTQPPSVSVAPGQTARISCSGDNLRHYYVYWYQQKPGQAPVLVIYGD
SKRPSGIPERFSGSNSGNTATLTISGTQAEDEADYYCQTYTGGASLVFGG
GTKLTVLGQ;

Isatuximab; comprising the VH and VL sequences of SEQ ID NOs: X and X, respectively, described in U.S. Pat. No. 8,153,765. The VH and the VL of Isatuximab may be expressed as IgG1/κ.

SEQ ID NO 20:
QVQLVQSGAEVAKPGTSVKLSCKASGYTFTDYWMQWVKQRPGQGLEWIGT
IYPGDGDTGYAQKFQGKATLTADKSSKTVYMHLSSLASEDSAVYYCARGD
YYGSNSLDYWGQGTSVTVSS
SEQ ID NO: 21:
DIVMTQSHLSMSTSLGDPVSITCKASQDVSTVVAWYQQKPGQSPRRLIYS
ASYRYIGVPDRFTGSGAGTDFTFTISSVQAEDLAVYYCQQHYSPPYTFGG
GTKLEIK

Other exemplary anti-CD38 antibodies that may be used in the methods of the invention include those described in Int. Pat. Publ. No. WO05/103083, Intl. Pat. Publ. No. WO06/125640, Intl. Pat. Publ. No. WO07/042309, Intl. Pat. Publ. No. WO08/047242 or Intl. Pat. Publ. No. WO14/178820.

In some embodiments, AL is cardiac stage I, cardiac stage II or cardiac stage III.

In some embodiments, AL is relapsed or refractory.

AL diagnosis is performed by a physician according to guidelines available for example at National Comprehensive Cancer Network (www_nccn.org/professionals/physician_gls/_f_guidelines_asp #site). AL patients present damage in various organ systems due to accumulation of light chains and their misfolded intermediates as amyloid fibers in vital organs, causing organ dysfunction and mortality. Patients may have multiple organ systems affected at diagnosis; about one third of patients have more than 3 organs affected at diagnosis (Chaulagain and Comenzo Curr Hematol Malig Rep 8:291-8, 2013). AL prognosis involves cardiac staging as cardiac damage appears to be present in all AL patients at diagnosis even if the patient is asymptomatic (Palladini et al., Blood 116:3426-30, 2010; Kristen et al., Blood 116:2455-61, 2010). Based on the presence of one, two or both cardiac biomarkers N-terminal prohormone of brain natriuretic peptide (NT-proBNP) and troponin T, AL patients may be classified to cardiac stage I, II or III, see e.g. Comenzo et al., Leukemia 26:2317-25, 2012.

Current treatment options for AL are directed towards killing the light chain immunoglobulin secreting plasma cells, and include a combination of agents such as Velcade® (bortezomib), cyclophosphamide such as Cytoxan® or Neosar®, Alkeran® (melphalan), Thalomid® (thalidomide), Revlimid® (lenalidomide), or Pomalyst® (pomalidomide) as well as steroids (dexamethasone), interferon alpha (IFN-α) and stem cell transplantation. High-dose Alkeran® (melphalan) may be used with stem cell transplantation (see for example Anderson et al., Systemic light chain amyloidosis, NCCN Clinical Practice Guidelines in Oncology Version I.2015, NCCN.org. 2014). NINLARO® (ixazomib), a proteasome inhibitor, is being evaluated for treatment of AL. NEOD001, a monoclonal antibody targeting AL amyloid protein is being evaluated for treatment of AL.

In some embodiments, MM is relapsed or refractory.

Currently available therapies for MM include chemotherapy, stem cell transplantation, Thalomid® (thalidomide), Revlimid® (lenalidomide), Velcade® (bortezomib), Kyprolis® (carfilzomib), Farydak® (panobinostat), Aredia® (pamidronate), and Zometa® (zoledronic acid). Current treatment protocols, which include a combination of chemotherapeutic agents such as Oncovin® (vincristine), BiCNU® (BCNU, carmustine), Alkeran® (melphalan), cyclophosphamide, Adriamycin® (doxorubicin), and prednisone or dexamethasone, yield a complete remission rate of only about 5%, and median survival is approximately 36-48 months from the time of diagnosis. Further, the investigational agent ixazomib has achieved positive results from a key clinical trial in relapsed multiple myeloma patients. Recent advances using high dose chemotherapy followed by autologous bone marrow or peripheral blood mononuclear cell transplantation have increased the complete remission rate and remission duration, yet overall survival has only been slightly prolonged, and no evidence for a cure has been obtained. Ultimately, all MM patients relapse, even under maintenance therapy with interferon-alpha (IFN-α) alone or in combination with steroids.

Various qualitative and/or quantitative methods may be used to determine relapse or refractory nature of the disease. Symptoms that may be associated with relapse or resistance are, for example, a decline or plateau of the well-being of the patient or re-establishment or worsening of various symptoms associated with hematological malignancy, and/or the spread of cancerous cells in the body from one location to other organs, tissues or cells. The symptoms associated with hematological malignancy may vary according to the type of cancer. For example, symptoms associated with AL may include increased fatigue, purpura, enlarged tongue, diarrhea or edema, proteinurea, or increased plasma free light chain.

In some embodiments, the HSCT is allogeneic, autologous or syngeneic, i.e. the donor is a twin. Autologous HSCT comprises the extraction of HSC from the subject and freezing of the harvested HSC. After myeloablation, the subject's stored HSC are transplanted into the subject. Allogeneic HSCT involves HSC obtained from an allogeneic HSC donor who has an HLA type that matches the subject.

“Hematopoietic stem cell transplantation” as used herein is the transplantation of blood stem cells derived from the bone marrow (in this case known as bone marrow transplantation), blood (such as peripheral blood and umbilical cord blood), or amniotic fluid.

“Undergoing hematopoietic stem cell transplantation” as used herein means that the patient did already receive, is receiving or will receive HSCT.

In some embodiments, the patient has completed chemotherapy and/or radiation therapy prior to HSCT.

Patients may be treated with chemotherapy and/or radiation therapy prior to HSCT (so-called pre-transplant preparation) to eradicate some or all of the patient's hematopoietic cells prior to transplant. The patient may also be treated with immunosuppressants in case of allogeneic HSCT. An exemplary pre-transplant preparation therapy is high-dose melphalan (see for example Skinner et al., Ann Intern Med 140:85-93, 2004; Gertz et al., Bone Marrow Transplant 34:1025-31, 2004; Perfetti et al., Haematologica 91:1635-43, 2006). The radiation therapy which may be employed in pre-transplant treatment may be carried out according to commonly known protocols in this field. Radiation therapy may also be provided simultaneously, sequentially or separately with the anti-CD38 antibody.

DARZALEX™ (daratumumab) may not mediate killing CD38⁺CD34⁺ stem cells within the transplant and hence is a suitable therapy to combine with HSCT. Antibodies competing with daratumumab, and/or antibodies binding the same epitope as DARZALEX™ (daratumumab) may also not kill CD38+CD34+ stem cells.

Other exemplary antibodies that can be used in the methods of the invention disclosed herein may not kill CD38⁺CD34⁺ stem cells within the transplant and hence are suitable therapies to combine with HSCT. Inability of the antibodies to kill CD38⁺CD34⁺ stem cells within the transplant can be assessed using the methods described herein.

Anti-CD38 antibodies used in the methods of the invention may also be selected de novo from, e.g., a phage display library, where the phage is engineered to express human immunoglobulins or portions thereof such as Fabs, single chain antibodies (scFv), or unpaired or paired antibody variable regions (Knappik et al., J Mol Biol 296:57-86, 2000; Krebs et al., J Immunol Meth 254:67-84, 2001; Vaughan et al., Nature Biotechnology 14:309-314, 1996; Sheets et al., PITAS (USA) 95:6157-6162, 1998; Hoogenboom and Winter, J Mol Biol 227:381, 1991; Marks et al., J Mol Biol 222:581, 1991). CD38 binding variable domains may be isolated from e.g., phage display libraries expressing antibody heavy and light chain variable regions as fusion proteins with bacteriophage pIX coat protein as described in Shi et al., J. Mol. Biol. 397:385-96, 2010 and Intl. Pat. Publ. No. WO09/085462). The antibody libraries may be screened for binding to human CD38 extracellular domain, obtained positive clones further characterized, Fabs isolated from the clone lysates, and subsequently cloned as full length antibodies. Such phage display methods for isolating human antibodies are established in the art. See for example: U.S. Pat. Nos. 5,223,409; 5,403,484; and 5,571,698, 5,427,908, 5,580,717, 5,969,108, 6,172,197, 5,885,793; 6,521,404; 6,544,731; 6,555,313; 6,582,915; and 6,593,081.

In some embodiments described herein, the anti-CD38 antibody does not mediate killing CD34-positive hematopoietic progenitor cells by complement dependent cytotoxicity (CDC).

“Does not kill” or “does not mediate killing of” refers to the inability of the anti-CD38 antibody to induce cell killing when compared to an appropriate control, such as an isotype control. The anti-CD38 antibody “does not kill” when the measured killing of cells in the presence of DARZALEX™ (daratumumab) is not statistically significant when compared to the killing of cells in the presence of an isotype control. Isotype control is a well-known term.

CD34-positive hematopoietic progenitor cell killing by CDC may be measured in fresh or frozen isolated CD34⁺ cells by incubating the cells in 10% serum with complement and 500 ng/ml anti-CD38 antibody, followed by assaying the degree of colony formation of the cells plated in semisolid medium according to known methods. For example, BFU-E and CFU-GM formation may be assessed after 14 days in culture using commercial reagents such as MethoCult™ by Stem Cell Technologies.

The Fc portion of the antibody may mediate antibody effector functions such as antibody-dependent cell-mediated cytotoxicity (ADCC), antibody-dependent cellular phagocytosis (ADCP) or complement dependent cytotoxicity (CDC). Such function may be mediated by binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components result in inhibition and/or depletion of target cells, for example CD38-expressing cells. Human IgG isotypes IgG1, IgG2, IgG3 and IgG4 exhibit differential capacity for effector functions. ADCC may be mediated by IgG1 and IgG3, ADCP may be mediated by IgG1, IgG2, IgG3 and IgG4, and CDC may be mediated by IgG1 and IgG3.

In some embodiments, the anti-CD38 antibody is of IgG1, IgG2, IgG3 or IgG4 isotype.

In some embodiments, the anti-CD38 antibody induces killing of CD38 expressing plasma cells in vitro by antibody-dependent cell-mediated cytotoxicity (ADCC), antibody-dependent cellular phagocytosis (ADCP), complement dependent cytotoxicity (CDC), apoptosis, or in vitro modulation of CD38 enzymatic activity.

In some embodiments, the anti-CD38 antibody induces killing of CD38 expressing cells in vitro by ADCC, ADCP or CDC.

In some embodiments, the anti-CD38 antibody induces killing of CD38 expressing cells in vitro by ADCC.

In some embodiments, the anti-CD38 antibody induces killing of CD38 expressing cells in vitro by ADCP.

In some embodiments, the anti-CD38 antibody induces killing of CD38 expressing cells in vitro by CDC.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells, monocytes, macrophages and neutrophils via Fc gamma receptors (FcγR) expressed on effector cells. For example, NK cells express FcγRIIIa, whereas monocytes express FcγRI, FcγRII and FcγRIIIa. Death of the antibody-coated target cell, such as CD38-expressing cells, occurs as a result of effector cell activity through the secretion of membrane pore-forming proteins and proteases. To assess ADCC activity of an anti-CD38 antibody, the antibody may be added to CD38-expressing cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Exemplary effector cells for such assays include peripheral blood mononuclear cells (PBMC) and NK cells. Exemplary target cells include Daudi cells (ATCC® CCL-213™) or B cell leukemia or lymphoma tumor cells expressing CD38. In an exemplary assay, target cells are labeled with 20 μCi of ⁵¹Cr for 2 hours and washed extensively. Cell concentration of the target cells can be adjusted to 1×10⁶ cells/ml, and anti-CD38 antibodies at various concentrations are added. Assays are started by adding Daudi cells at an effector:target cell ratio of 40:1. After incubation for 3 hr at 37° C., assays are stopped by centrifugation, and ⁵¹Cr release from lysed cells are measured in a scintillation counter. Percentage of cellular cytotoxicity may be calculated as % maximal lysis which may be induced by adding 3% perchloric acid to target cells. Anti-CD38 antibodies used in the methods of the invention may induce ADCC by about 20%, 25%, 30%, 35%, 40%, 45%, 50%, 55%, 60%, 65%, 70%, 75%, 80%, 85%, 90%, 95% or 100% of control (cell lysis induced by 3% perchloric acid).

“Antibody-dependent cellular phagocytosis” (“ADCP”) refers to a mechanism of elimination of antibody-coated target cells by internalization by phagocytic cells, such as macrophages or dendritic cells. ADCP may be evaluated by using monocyte-derived macrophages as effector cells and Daudi cells (ATCC® CCL-213™) or B cell leukemia or lymphoma tumor cells expressing CD38 as target cells engineered to express GFP or other labeled molecule. Effctor:target cell ratio may be for example 4:1. Effector cells may be incubated with target cells for 4 hours with or without anti-CD38 antibody. After incubation, cells may be detached using accutase. Macrophages can be identified with anti-CD11b and anti-CD14 antibodies coupled to a fluorescent label, and percent phagocytosis can be determined based on % GFP fluorescent in the CD11⁺CD14⁺ macrophages using standard methods. Anti-CD38 antibodies used in the methods of the invention may induce ADCP by about 20%, 25%, 30%, 35%, 40%, 45%, 50%, 55%, 60%, 65%, 70%, 75%, 80%, 85%, 90%, 95% or 100%.

“Complement-dependent cytotoxicity”, or “CDC”, refers to a mechanism for inducing cell death in which an Fc effector domain of a target-bound antibody binds and activates complement component C1q which in turn activates the complement cascade leading to target cell death. Activation of complement may also result in deposition of complement components on the target cell surface that facilitate ADCC by binding complement receptors (e.g., CR3) on leukocytes. CDC of CD38-expressing cells can be measured for example by plating Daudi cells at 1×10⁵ cells/well (50 μl/well) in RPMI-B (RPMI supplemented with 1% BSA), adding 50 μl anti-CD38 antibodies to the wells at final concentration between 0-100 μg/ml, incubating the reaction for 15 min at room temperature, adding 11 μl of pooled human serum to the wells, and incubation the reaction for 45 min at 37° C. Percentage (%) lysed cells may be detected as % propidium iodide stained cells in FACS assay using standard methods. Anti-CD38 antibodies used in the methods of the invention may induce CDC by about 20%, 25%, 30%, 35%, 40%, 45%, 50%, 55%, 60%, 65%, 70%, 75%, 80%, 85%, 90%, 95% or 100%.

The ability of monoclonal antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such mAbs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-40, 2002), application of a variant CHO line EB66 as the host cell line (Olivier et al., MAbs 2 (4), 2010; Epub ahead of print; PMID: 20562582), application of a rat hybridoma cell line YB2/0 as the host cell line (Shinkawa et al., J Biol Chem 278:3466-73, 2003), introduction of small interfering RNA specifically against the α 1,6-fucosyltrasferase (FUT8) gene (Mori et al., Biotechnol Bioeng 88:901-8, 2004), or coexpression of β-1,4-N-acetylglucosaminyltransferase III and Golgi α-mannosidase II or a potent alpha-mannosidase I inhibitor, kifunensine (Ferrara et al., J Biol Chem 281:5032-6, 2006,; Ferrara et al., Biotechnol Bioeng 93:851-61, 2006; Xhou et al., Biotechnol Bioeng 99:652-65, 2008). ADCC elicited by anti-CD38 antibodies used in the methods of the invention, and in some embodiments of each and every one of the numbered embodiments listed below, may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056. CDC elicited by anti-CD38 antibodies used in the methods of the invention, and in some embodiments of each and every one of the numbered embodiments listed below, may also be enhanced by certain substitutions in the antibody Fc. Exemplary substitutions are, for example, substitutions at amino acid positions 423, 268, 267 and/or 113 (residue numbering according to the EU index) as described in Moore et al., Mabs 2:181-9, 2010.

In some embodiments, the anti-CD38 antibody comprises a substitution in the antibody Fc.

In some embodiments, the anti-CD38 antibody comprises a substitution in the antibody Fc at amino acid positions 256, 290, 298, 312, 356, 330, 333, 334, 360, 378 and/or 430 (residue numbering according to the EU index).

In some embodiments, the anti-CD38 antibody comprises a substitution in the antibody Fc at amino acid position 113, 267, 268 and/or 423 (residue numbering according to the EU index).

In some embodiments, the anti-CD38 antibody comprises has a biantennary glycan structure with fucose content of about between 0% to about 15%, for example 15%, 14%, 13%, 12%, 11% 10%, 9%, 8%, 7%, 6%, 5%, 4%, 3%, 2%, 1% or 0%.

In some embodiments, the anti-CD38 antibody comprises has a biantennary glycan structure with fucose content of about 50%, 40%, 45%, 40%, 35%, 30%, 25%, 20%, 15%, 14%, 13%, 12%, 11% 10%, 9%, 8%, 7%, 6%, 5%, 4%, 3%, 2%, 1% or 0%

Substitutions in the Fc and reduced fucose content may enhance the ADCC activity of the anti-CD38 antibody.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Intl. Pat. Publ. No. WO2008/077546; 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS) or 5) separation of the mAb oligosaccharides from the mAb protein by specific enzymatic deglycosylation with PNGase F at Asn 297. The oligosaccharides released can be labeled with a fluorophore, separated and identified by various complementary techniques which allow: fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosacharride forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” as used in the application refers to antibodies with fucose content of about 0%-15%.

“Normal fucose” or ‘normal fucose content” as used herein refers to antibodies with fucose content of about over 50%, typically about over 60%, 70%, 80% or over 85%.

The anti-CD38 antibody used in the methods of the invention may induce killing of CD38-expressing cells by apoptosis in vitro. Methods for evaluating apoptosis are well known, and include for example annexin IV staining using standard methods. The anti-CD38 antibody in the methods of the invention may induce apoptosis in about 20%, 25%, 30%, 35%, 40%, 45%, 50%, 55%, 60%, 65%, 70%, 75%, 80%, 85%, 90%, 95% or 100% of cells.

The anti-CD38 antibody used in the methods of the invention may induce in vitro killing of CD38-expressing cells by modulation of CD38 enzymatic activity. CD38 is a multifunctional ectoenzme with ADP-ribosyl cyclase 1 activity catalyzing the formation of cyclic ADP-ribose (cADPR) and ADPR from NAD⁺, and also functions to hydrolyze NAD⁺ and cADPR to ADPR. CD38 also catalyzes the exchange of the nicotinamide group of NADP⁺ with nicotinic acid under acidic conditions, to yield NAADP⁺ (nicotinic acid-adenine dinucleotide phosphate). Modulation of the enzymatic activity of human CD38 with anti-CD38 antibodies used in the methods of the invention may be measured in an assay described in Graeff et al., J. Biol. Chem. 269:30260-7, 1994). For example, substrate NGD⁺ may be incubated with CD38, and the modulation of the production of cyclic GDP-ribose (cGDPR) may be monitored spectrophotometrically at excitation at 340 nM and emission at 410 nM at different time points after addition of the antibody at various concentrations. Inhibition of the synthesis of cADPR may be determined according to the HPLC method described in Munshi et al., J. Biol. Chem. 275:21566-71, 2000. The anti-CD38 antibodies used in the methods of the invention may inhibit CD38 enzymatic activity by at least about 20%, 25%, 30%, 35%, 40%, 45%, 50%, 55%, 60%, 65%, 70%, 75%, 80%, 85%, 90%, 95% or 100%.

Antibodies that are substantially identical to the antibody comprising the VH of SEQ ID NO: 4 and the VL of SEQ ID NO: 5 may be used in the methods of the invention. The term “substantially identical” means that the antibody VH or VL amino acid sequences being compared are identical or have “insubstantial differences”. Insubstantial differences are substitutions of 1, 2, 3, 4, 5, 6, 7, 8, 9, 10, 11, 12, 13, 14, or 15 amino acids in the antibody VL and/or VL that do not adversely affect antibody properties. Percent identity may be determined for example by pairwise alignment using the default settings of the AlignX module of Vector NTI v.9.0.0 (Invitrogen, Carlsbad, CA). The protein sequences of the present invention may be used as a query sequence to perform a search against public or patent databases to, for example, identify related sequences. Exemplary programs used to perform such searches are the XBLAST or BLASTP programs (www_ncbi_nlm/nih_gov), or the GenomeQuest™ (GenomeQuest, Westborough, MA) suite using the default settings. Exemplary substitutions that may be made to the antibodies that specifically bind CD38 used in the methods of the invention are for example conservative substitutions with an amino acid having similar charge, hydrophobic, or stereochemical characteristics. Conservative substitutions may also be made to improve antibody properties, for example stability or affinity, or to improve antibody effector functions. 1, 2, 3, 4, 5, 6, 7, 8, 9, 10, 11, 12, 13, 14, or 15 amino acid substitutions may be made for example to the VH and/or the VL of the anti-CD38. Furthermore, any native residue in the heavy or light chain may also be substituted with alanine, as has been previously described for alanine scanning mutagenesis (MacLennan et al., Acta Physiol Scand Suppl 643:55-67, 1998; Sasaki et al., Adv Biophys 35:1-24, 1998). Desired amino acid substitutions may be determined by those skilled in the art at the time such substitutions are desired. Amino acid substitutions may be done for example by PCR mutagenesis (U.S. Pat. No. 4,683,195). Libraries of variants may be generated using well known methods, for example using random (NNK) or non-random codons, for example DVK codons, which encode 11 amino acids (Ala, Cys, Asp, Glu, Gly, Lys, Asn, Arg, Ser, Tyr, Trp) and screening the libraries for variants with desired properties. The generated variants may be tested for their binding to CD38, their ability to induce ADCC, ADCP or apoptosis, or modulate CD38 enzymatic activity in vitro using methods described herein.

“Conservative modifications” refer to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., (1988) Acta Physiol Scand Suppl 643:55-67; Sasaki et al., (1988) Adv Biophys 35:1-24). Amino acid substitutions to the antibodies of the invention may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

In some embodiments, the antibody may bind CD38 with a dissociation constant (K_{D})) of less than about 1×10⁻⁷ M, 1×10⁻⁸ M, 1×10⁻⁹ M, 1×10⁻¹⁰ M, 1×10⁻¹¹ M, 1×10⁻¹² M, 1×10⁻¹³ M, 1×10⁻¹⁴ M or 1×10⁻¹⁵, as determined by surface plasmon resonance or the Kinexa method, as practiced by those of skill in the art. In some embodiments, the antibody binds human CD38 with a K_{D} of less than about 1×10⁻⁸ M. In some embodiments, the antibody binds human CD38 with a K_{D} of less than about 1×10⁻⁹ M.

KinExA instrumentation, ELISA or competitive binding assays known to those skilled in the art. The measured affinity of a particular antibody/CD38 interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer, such as the buffer described herein. Skilled in the art will appreciate that the internal error for affinity measurements for example using Biacore 3000 or ProteOn (measured as standard deviation, SD) may typically be within 5-33% for measurements within the typical limits of detection. Therefore the term “about” in the context of K_{D} reflects the typical standard deviation in the assay. For example, the typical SD for a K_{D} of 1×10⁻⁹ M is up to ±0.33×10⁻⁹ M.

In some embodiments, the anti-CD38 antibody is a bispecific antibody. The VL and/or the VH regions of the existing anti-CD38 antibodies or the VL and VH regions identified de novo as described above may be engineered into bispecific full length antibodies. Such bispecific antibodies may be made by modulating the CH3 interactions between the monospecific antibody heavy chains to form bispecific antibodies using technologies such as those described in U.S. Pat. No. 7,695,936; Intl. Pat. Publ. No. WO04/111233; U.S. Pat. Publ. No. US2010/0015133; U.S. Pat. Publ. No. US2007/0287170; Intl. Pat. Publ. No. WO2008/119353; U.S. Pat. Publ. No. US2009/0182127; U.S. Pat. Publ. No. US2010/0286374; U.S. Pat. Publ. No. US2011/0123532; Intl. Pat. Publ. No. WO2011/131746; Int. Pat. Publ. No. WO2011/143545; or U.S. Pat. Publ. No. US2012/0149876. Additional bispecific structures into which the VL and/or the VH regions of the antibodies of the invention may be incorporated are for example Dual Variable Domain Immunoglobulins (Inlt. Pat. Publ. No. WO2009/134776), or structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441).

For example, bispecific antibodies may be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl.Pat. Publ. No. WO2011/131746. In the methods, the first monospecific bivalent antibody (e.g., anti-CD38 antibody) and the second monospecific bivalent antibody are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl) phosphine (TCEP), L-cysteine and beta-mercaptoethanol, preferably a reducing agent selected from the group consisting of: 2-mercaptoethylamine, dithiothreitol and tris(2-carboxyethyl) phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Exemplary CH3 mutations that may be used in a first heavy chain and in a second heavy chain of the bispecific antibody are K409R and/or F405L.

In some embodiments, the anti-CD38 antibody is conjugated to a toxin. Conjugation methods and suitable toxins are well known.

In some embodiments, the subject having AL is homozygous for phenylalanine at position 158 of CD16 (FcγRIIIa-158F/F genotype) or heterozygous for valine and pheynylalanine at position 158 of CD16 (FcγRIIIa-158F/V genotype). CD16 is also known as the Fc gamma receptor Illa (FcγRIIIa) or the low affinity immunoglobulin gamma Fc region receptor III-A isoform. Valine/phenylalanine (V/F) polymorphism at FcγRIIIa protein residue position 158 has been shown to affect FcγRIIIa affinity to human IgG. Receptor with FcγRIIIa-158F/F or FcγRIIIa-158F/V polymorphisms demonstrates reduced Fc engagement and therefore reduced ADCC when compared to the FcγRIIIa-158V/V. The lack of or low amount of fucose on human N-linked oligosaccharides improves the ability of the antibodies to induce ADCC due to improved binding of the antibodies to human FcγRIIIa (CD16) (Shields et al., J Biol Chem 277:26733-40, 2002). Patients can be analyzed for their FcγRIIIa polymorphism using routine methods.

The invention also provides for a method of treating a subject having AL, comprising administering to the patient in need thereof an anti-CD38 antibody that binds to the region SKRNIQFSCKNIYR (SEQ ID NO: 2) and the region EKVQTLEAWVIHGG (SEQ ID NO: 3) of human CD38 (SEQ ID NO: 1), wherein the anti-CD38 antibody induces in vitro killing of CD38-expressing pathogenic plasma cells by antibody-dependent cell-mediated cytotoxicity (ADCC), antibody-dependent cellular phagocytosis (ADCP), complement dependent cytotoxicity (CDC), apoptosis, or in vitro modulation of CD38 enzymatic activity, wherein the subject is homozygous for valine at position 158 of CD16.

The invention also provides for a method of treating a subject having AL, comprising administering to the patient in need thereof an anti-CD38 antibody that binds to the region SKRNIQFSCKNIYR (SEQ ID NO: 2) and the region EKVQTLEAWVIHGG (SEQ ID NO: 3) of human CD38 (SEQ ID NO: 1), wherein the anti-CD38 antibody induces in vitro killing of CD38-expressing pathogenic plasma cells by antibody-dependent cell-mediated cytotoxicity (ADCC), antibody-dependent cellular phagocytosis (ADCP), complement dependent cytotoxicity (CDC), apoptosis, or in vitro modulation of CD38 enzymatic activity, wherein the subject is homozygous for phenylalanine at position 158 of CD16 or heterozygous for valine and pheynylalanine at position 158 of CD16.

The invention also provides for a method of treating a patient having AL, comprising administering to the patient in need thereof an anti-CD38 antibody that competes for binding to CD38 with an antibody comprising a heavy chain variable region (VH) of SEQ ID NO: 4 and a light chain variable region (VL) of SEQ ID NO: 5, wherein the anti-CD38 antibody induces in vitro killing of CD38-positive pathogenic plasma cells by antibody-dependent cell-mediated cytotoxicity (ADCC), antibody-dependent cellular phagocytosis (ADCP), complement dependent cytotoxicity (CDC), apoptosis, or in vitro modulation of CD38 enzymatic activity, wherein the patient is homozygous for valine at position 158 of CD16.

The invention also provides for a method of treating a patient having AL, comprising administering to the patient in need thereof an anti-CD38 antibody that competes for binding to CD38 with an antibody comprising a heavy chain variable region (VH) of SEQ ID NO: 4 and a light chain variable region (VL) of SEQ ID NO: 5, wherein the anti-CD38 antibody induces in vitro killing of CD38-positive pathogenic plasma cells by antibody-dependent cell-mediated cytotoxicity (ADCC), antibody-dependent cellular phagocytosis (ADCP), complement dependent cytotoxicity (CDC), apoptosis, or in vitro modulation of CD38 enzymatic activity, wherein the patient is homozygous for phenylalanine at position 158 of CD16 or heterozygous for valine and pheynylalanine at position 158 of CD16.

The invention also provides for the method of treating a patient having AL, comprising
determining that the patient is homozygous or heterozygous for valine at position 158 of CD16; and
administering to the patient an anti-CD38 antibody
that competes for binding to CD38 with an antibody comprising a heavy chain variable region (VH) of SEQ ID NO: 4 and a light chain variable region (VL) of SEQ ID NO: 5;
comprises the heavy chain complementarity determining regions (HCDR) 1 (HCDR1), 2 (HCDR2) and 3 (HCDR3) sequences of SEQ ID NOs: 6, 7 and 8, respectively, and the light chain complementarity determining regions (LCDR) 1 (LCDR1), 2 (LCDR2) and 3 (LCDR3) sequences of SEQ ID NOs: 9, 10 and 11, respectively; or
comprises the heavy chain variable region (VH) of SEQ ID NO: 4 and the light chain variable region (VL) of SEQ ID NO: 5 for a time sufficient to treat the patient.

In some embodiments, the determining that the patient is homozygous or heterozygous for valine at position 158 of CD16 is done by polymerase chain reaction (PCR) and sequencing.

### Administration/Pharmaceutical Compositions

In the methods of the invention, the anti-CD38 antibody may be provided in suitable pharmaceutical compositions comprising the anti-CD38 antibody and a pharmaceutically acceptable carrier. The carrier may be diluent, adjuvant, excipient, or vehicle with which the anti-CD38 antibody is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc.

The concentration of the anti-CD38 antibody in such pharmaceutical formulation may vary widely, i.e., from less than about 0.5%, usually to at least about 1% to as much as 15 or 20%, 25%, 30%, 35%, 40%, 45% or 50% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, see especially pp. 958-989.

The mode of administration of the anti-CD38 antibody may be any suitable route such as parenteral administration, e.g., intradermal, intramuscular, intraperitoneal, intravenous or subcutaneous, pulmonary, transmucosal (oral, intranasal, intravaginal, rectal) or other means appreciated by the skilled artisan, as well known in the art.

The anti-CD38 antibody in the methods of the invention may be administered to a patient by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours.

The dose of the anti-CD38 antibody given to a patient is sufficient to alleviate or at least partially arrest the disease being treated (“therapeutically effective amount”) and may be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat AL, but 9, 10, 11, 12, 13, 14, 15, 16, 17, 18, 19, 20 or more doses may be given.

The administration of the anti-CD38 antibody in the methods of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the anti-CD38 antibody may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

In some embodiments, the anti-CD38 antibody is administered at 16 mg/kg once a week for 8 weeks, followed by administration at 16 mg/kg once every two weeks for 16 weeks, followed by administration at 16 mg/kg once every four weeks until discontinuation.

In some embodiments, the anti-CD38 antibody is administered at 8 mg/kg once a week for 8 weeks, followed by administration at 8 mg/kg once every two weeks for 16 weeks, followed by administration at 8 mg/kg once every four weeks until discontinuation.

In some embodiments, the anti-CD38 antibody is administered at 16 mg/kg once a week for 4 weeks, followed by administration at 16 mg/kg once every two weeks for 16 weeks, followed by administration at 16 mg/kg once every four weeks until discontinuation.

In some embodiments, the anti-CD38 antibody is administered at 8 mg/kg once a week for 4 weeks, followed by administration at 8 mg/kg once every two weeks for 16 weeks, followed by administration at 8 mg/kg once every four weeks until discontinuation.

The anti-CD38 antibody may be administered as maintenance therapy, such as, e.g., once a week for a period of 6 months or more.

For example, the anti-CD38 antibody may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The anti-CD38 antibody may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The anti-CD38 antibody may be lyophilized for storage and reconstituted in a suitable carrier prior to use. This technique has been shown to be effective with conventional protein preparations and well known lyophilization and reconstitution techniques can be employed.

Subcutaneous Administration of Pharmaceutical Compositions Comprising an Antibody that Specifically Binds Cd38 and a Hyaluronidase

The anti-CD38 antibody may be administered as a pharmaceutical composition comprising the anti-CD38 antibody and a hyaluronidase subcutaneously.

The concentration of the anti-CD38 antibody in the pharmaceutical composition administered subcutaneously may be about 20 mg/ml.

The pharmaceutical composition administered subcutaneously may comprise between about 1,200 mg-1,800 mg of the anti-CD38 antibody.

The pharmaceutical composition administered subcutaneously may comprise about 1,200 mg of the anti-CD38 antibody.

The pharmaceutical composition administered subcutaneously may comprise about 1,600 mg of the anti-CD38 antibody.

The pharmaceutical composition administered subcutaneously may comprise about 1,800 mg of the anti-CD38 antibody.

The pharmaceutical composition administered subcutaneously may comprise between about 30,000 U-45,000 U of the hyaluronidase.

The pharmaceutical composition administered subcutaneously may comprise about 1,200 mg of the anti-CD38 antibody and about 30,000 U of the hyaluronidase.

The pharmaceutical composition administered subcutaneously may comprise about 1,800 mg of the anti-CD38 antibody and about 45,000 U of the hyaluronidase.

The pharmaceutical composition administered subcutaneously may comprise about 1,600 mg of the anti-CD38 antibody and about 30,000 U of the hyaluronidase.

The pharmaceutical composition administered subcutaneously may comprise about 1,600 mg of the anti-CD38 antibody and about 45,000 U of the hyaluronidase.

The pharmaceutical composition administered subcutaneously may comprise the hyaluronidase rHuPH20 having the amino acid sequence of SEQ ID NO: 22.

rHuPH20 is a recombinant hyaluronidase (HYLENEX® recombinant) and is described in Int. Pat. Publ. No. WO2004/078140.

Hyaluronidase is an enzyme that degrades hyaluronic acid (EC 3.2.1.35) and lowers the viscosity of hyaluronan in the extracellular matrix, thereby increasing tissue permeability.

SEQ ID NO: 22
MGVLKFKHIFFRSFVKSSGVSQIVFTFLLIPCCLTLNFRAPPVIPNVPFL
WAWNAPSEFCLGKFDEPLDMSLFSFIGSPRINATGQGVTIFYVDRLGYYP
YIDSITGVTVNGGIPQKISLQDHLDKAKKDITFYMPVDNLGMAVIDWEEW
RPTWARNWKPKDVYKNRSIELVQQQNVQLSLTEATEKAKQEFEKAGKDFL
VETIKLGKLLRPNHLWGYYLFPDCYNEIHYKKPGYNGSCFNVEIKRNDDL
SWLWNESTALYPSIYLNTQQSPVAATLYVRNRVREAIRVSKIPDAKSPLP
VFAYTRIVFTDQVLKFLSQDELVYTFGETVALGASGIVIWGTLSIMRSMK
SCLLLDNYMETILNPYIINVTLAAKMCSQVLCQEQGVCIRKNWNSSDYLH
LNPDNFAIQLEKGGKFTVRGKPTLEDLEQFSEKFYCSCYSTLSCKEKADV
KDTDAVDVCIADGVCIDAFLKPPMETEEPQIFYNASPSTLSATMFIVSIL
FLIISSVASL

The administration of the pharmaceutical composition comprising the anti-CD38 antibody and the hyaluronidase may be repeated after one day, two days, three days, four days, five days, six days, one week, two weeks, three weeks, four weeks, five weeks, six weeks, seven weeks, two months, three months, four months, five months, six months or longer. Repeated courses of treatment are also possible, as is chronic administration. The repeated administration may be at the same dose or at a different dose. For example, the pharmaceutical composition comprising the anti-CD38 antibody and the hyaluronidase may be administered once weekly for eight weeks, followed by once in two weeks for 16 weeks, followed by once in four weeks. The pharmaceutical compositions to be administered may comprise about 1,200 mg of the anti-CD38 antibody and about 30,000 U of hyaluronidase, wherein the concentration of the antibody that specifically binds CD38 in the pharmaceutical composition is about 20 mg/ml. The pharmaceutical compositions to be administered may comprise about 1,800 mg of the anti-CD38 antibody and about 45,000 U of hyaluronidase. The pharmaceutical compositions to be administered may comprise about 1,600 mg of the anti-CD38 antibody and about 30,000 U of hyaluronidase. The pharmaceutical compositions to be administered may comprise about 1,600 mg of the anti-CD38 antibody and about 45,000 U of hyaluronidase.

The pharmaceutical composition comprising the anti-CD38 antibody and the hyaluronidase may be administered subcutaneously to the abdominal region.

The pharmaceutical composition comprising the anti-CD38 antibody and the hyaluronidase may be administered in a total volume of about 80 ml, 90 ml, 100 ml, 110 ml or 120 ml.

For administration, 20 mg/ml of the anti-CD38 antibody in 25 mM sodium acetate, 60 mM sodium chloride, 140 mM D-mannitol, 0.04% polysorbate 20, pH 5.5 may be mixed with rHuPH20, 1.0 mg/mL (75-150 kU/mL) in 10 mM L-Histidine, 130 mM NaCl, 10 mM L-Methionine, 0.02% Polysorbate 80, pH 6.5 prior to administration of the mixture to a subject.

### Combination Therapies

The anti-CD38 antibody may be administered in combination with a second therapeutic agent.

The invention also provides for a method of treating light chain amyloidosis (AL), comprising administering to a patient in need thereof an anti-CD38 antibody in combination with a proteasome inhibitor for a time sufficient to treat AL.

The invention also provides for a method of treating light chain amyloidosis (AL), comprising administering to a patient in need thereof an anti-CD38 antibody in combination with a proteasome inhibitor and a corticosteroid for a time sufficient to treat AL.

The invention also provides for a method of treating light chain amyloidosis (AL), comprising administering to a patient in need thereof an anti-CD38 antibody in combination with a proteasome inhibitor, a corticosteroid and a cyclophosphamide for a time sufficient to treat AL.

In some embodiments, the second therapeutic agent is a proteasome inhibitor.

In some embodiments, the proteasome inhibitor is Velcade® (bortezomib), or vinca alkaloids, for example vincristine or an anthracycline, such as doxorubicin.

In some embodiments, the second therapeutic agent is a corticosteroid.

In some embodiments, the corticosteroid is dexamethasone.

In some embodiments, the corticosteroid is prednisone.

In some embodiments, the second therapeutic agent is a cyclophosphamide.

In some embodiments, the second therapeutic agent is a glutamic acid derivative.

In some embodiments, the glutamic acid derivative is Thalomid® (thalidomide), Revlimid® (lenalidomide), Actimid® (CC4047).

In some embodiments, the second therapeutic agent is Velcade® (bortezomib), cyclophosphamide such as Cytoxan® or Neosar®, Alkeran® (melphalan), Thalomid® (thalidomide), Revlimid® (lenalidomide), or Pomalyst® (pomalidomide), a corticosteroid (dexamethasone), interferon alpha (IFN-α), stem cell transplantation, Ninlaro® (ixazomib) or NEOD001.

Bortezomib may be administered at 1.3 mg/m² SQ twice weekly or once weekly

Cyclophosphamide may be administered IV (intermittent therapy) 40-50 mg/kg (400-1800 mg/m²) divided over 2-5 days; may be repeated at intervals of 2-4 weeks; IV (continuous daily therapy): 60-120 mg/m²/day (1-2.5 mg/kg/day);

PO (intermittent therapy): 400-1000 mg/m² divided over 4-5 days or

PO (continuous daily therapy): 50-100 mg/m²/day or 1-5 mg/kg/day.

Dexamethasone may be administered 40 mg/week, or 20 mg pre- and post-dose with the anti-CD38 antibody.

Melphalan may be administered 9 mg/m², orally, once daily on Days 1 to 4 of each cycle up to Cycle 9.

Thalidomide may be administered 200 mg orally once daily.

Lenalidomide may be administered 25 mg/day orally on days 1-21 for each cycle.

Pomalidomide may be administered 4 mg orally on days 1-21 of repeated 28-day cycle.

Ixazomib may be administered at 24 mg/kg IV every 28 days.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib and cyclophosphamide for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib, cyclophosphamide and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib, cyclophosphamide and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with melphalan for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib and melphalan for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib, melphalan and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with bortezomib, melphalan and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with IFN-α for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with IFN-α and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with IFN-α and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with lenalinomide for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with lenalinomide and cyclophosphamide for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with lenalinomide, cyclophosphamide and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with lenalinomide, cyclophosphamide and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with pomalinomide for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with pomalinomide and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with pomalinomide and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with thalidomide for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with thalidomide and a corticosteroid for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with thalidomide and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the LCDR1, the LCDR3 and the LCDR3 of SEQ ID NOs: 9, 10 and 11, respectively, in combination with ixazomib for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib for a time sufficient to treat light chain amyloidosis (AL) for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib and cyclophosphamide for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib, cyclophosphamide and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib, cyclophosphamide and dexamethasone for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib and dexamethasone for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with melphalan for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib and melphalan for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib, melphalan and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with bortezomib, melphalan and dexamethasone for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with IFN-α for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with IFN-α and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with IFN-α and dexamethasone for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with lenalinomide for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with lenalinomide and cyclophosphamide for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with lenalinomide, cyclophosphamide and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with lenalinomide, cyclophosphamide and dexamethasone for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with pomalinomide for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with pomalinomide and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with pomalinomide and dexamethasone for a time sufficient to treat AL.

The invention also provides for a method of treating light chain amyloidosis (AL), comprising administering to a patient in need thereof an anti-CD38 antibody comprising the HCDR1, the HCDR2 and the HCDR3 of SEQ ID NOs: 6, 7 and 8, respectively and the VH of SEQ ID NO: 4 and the VL of SEQ ID NO: 5 in combination with thalidomide for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with thalidomide and a corticosteroid for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with thalidomide and dexamethasone for a time sufficient to treat AL.

The invention also provides for a method of treating light chain amyloidosis (AL), comprising administering to a patient in need thereof an anti-CD38 antibody comprising the VH of SEQ ID NO: 4 and the VL of SEQ ID NO: 5 in combination with ixazomib for a time sufficient to treat AL.

In some embodiments, the patient is resistant to treatment with a proteasome inhibitor.

In some embodiments, the patient is resistant to treatment with cyclophosphamide.

In some embodiments, the patient is resistant to treatment with a corticosteroid.

In some embodiment, the patient is resistant to treatment with a proteasome inhibitor, cyclophosphamide and a corticosteroid.

In some embodiment, the patient is resistant to treatment with a Velcade® (bortezomib), cyclophosphamide and dexamethasone.

In some embodiments, the combination of the anti-CD38 antibody and the second therapeutic agent may be administered over any convenient timeframe. For example, the anti-CD38 antibody and the second therapeutic agent may be administered to a patient on the same day, and even in the same intravenous infusion. However, the anti-CD38 antibody and the second therapeutic agent may also be administered on alternating days or alternating weeks or months, and so on. In some embodiments, the anti-CD38 antibody and the second therapeutic agent may be administered with sufficient proximity in time that they are simultaneously present (e.g., in the serum) at detectable levels in the patient being treated. In some embodiments, an entire course of treatment with the anti-CD38 antibody consisting of a number of doses over a time period is followed or preceded by a course of treatment with the second therapeutic agent, consisting of a number of doses. A recovery period of 1, 2 or several days or weeks may be used between administration of the anti-CD38 antibody and the second therapeutic agent.

Anti-CD38 antibody or a combination of anti-CD38 antibody and the second therapeutic agent may be administered together with any form of radiation therapy including external beam radiation, intensity modulated radiation therapy (IMRT) and any form of radiosurgery including Gamma Knife, Cyberknife, Linac, and interstitial radiation (e.g. implanted radioactive seeds, GliaSite balloon), and/or with surgery.

While having described the invention in general terms, the embodiments of the invention will be further disclosed in the following examples that should not be construed as limiting the scope of the claims.

### Further Embodiments of the Invention

Set out below are certain further embodiments of the invention according to the disclosures elsewhere herein. Features from embodiments of the invention set out above described as relating to the invention disclosed herein also relate to each and every one of these further numbered embodiments.
1. An anti-CD38 antibody that competes for binding to CD38 with an antibody comprising a heavy chain variable region (VH) of SEQ ID NO: 4 and a light chain variable region (VL) of SEQ ID NO: 5 for use in treating a patient having a CD38-positive hematological malignancy, wherein the patient is undergoing hematopoietic stem cell transplantation (HSCT).
2. The anti-CD38 antibody for use according to embodiment 1, wherein the CD38-positive hematological malignancy is
   a. light chain amyloidosis (AL), multiple myeloma (MM), acute lymphoblastic leukemia (ALL), non-Hodgkin's lymphoma (NHL), diffuse large B-cell lymphoma (DLBCL), Burkitt's lymphoma (BL), follicular lymphoma (FL) or mantle-cell lymphoma (MCL);
   b. a plasma cell disease;
   c. light chain amyloidosis (AL);
   d. multiple myeloma (MM); or
   e. Waldenstrom's macroglobulinemia.
3. The anti-CD38 antibody for use according to embodiment 2, wherein AL is cardiac stage I, cardiac stage II, cardiac stage III, relapsed or refractory.
4. The anti-CD38 antibody for use according to any one of embodiments 1-3, wherein the HSCT
   a. is allogeneic, autologous or syngeneic; or
   b. comprises transplantation of blood stem cells derived from bone marrow, blood or amniotic fluid.
5. The anti-CD38 antibody for use according to any one of embodiments 1-4, wherein the anti-CD38 antibody is administered prior to, during or after HSCT.
6. The anti-CD38 antibody for use according to any one of embodiments 1-5, wherein the patient has completed chemotherapy and/or radiation therapy prior to HSCT.
7. The anti-CD38 antibody for use according to any one of embodiments 1-6, wherein the anti-CD38 antibody binds to the region SKRNIQFSCKNIYR (SEQ ID NO: 2) and the region EKVQTLEAWVIHGG (SEQ ID NO: 3) of human CD38 (SEQ ID NO: 1).
8. The anti-CD38 antibody for use according to any one of embodiments 1-7, wherein the anti-CD38 antibody comprises the heavy chain complementarity determining regions (HCDR) 1, HCDR2 and HCDR3 sequences of SEQ ID NOs: 6, 7 and 8, respectively; and the light chain complementarity determining regions (LCDR) 1, LCDR2 and LCDR3 sequences of SEQ ID NOs: 9, 10 and 11, respectively.
9. The anti-CD38 antibody for use according to any one of embodiments 1-8, wherein the anti-CD38 antibody comprises the heavy chain variable region (VH) of SEQ ID NO: 4 and the light chain variable region (VL) of SEQ ID NO: 5.
10. The anti-CD38 antibody for use according to any one of embodiments 1-9, wherein the anti-CD38 antibody comprises a heavy chain comprising an amino acid sequence that is 95%, 96%, 97%, 98% or 99% identical to that of SEQ ID NO: 12 and a light chain comprising an amino acid sequence that is 95%, 96%, 97%, 98% or 99% identical to that of SEQ ID NO: 13.
11. The anti-CD38 antibody for use according to any one of embodiments 1-10, wherein the anti-CD38 antibody comprises a heavy chain comprises the heavy chain of SEQ ID NO: 12 and the light chain of SEQ ID NO: 13.
12. An anti-CD38 antibody that competes for binding to CD38 with an antibody comprising a heavy chain variable region (VH) of SEQ ID NO: 4 and a light chain variable region (VL) of SEQ ID NO: 5 for use in treating a patient having light chain amyloidosis.
13. The anti-CD38 antibody for use according to embodiment 12, wherein the patient is resistant to treatment with a proteasome inhibitor, cyclophosphamide and/or a corticosteroid.
14. The anti-CD38 antibody for use according to embodiment 13, wherein the proteasome inhibitor is Velcade® (bortezomib).
15. The anti-CD38 antibody for use according to any one of embodiments 13-14, wherein the corticosteroid is dexamethasone.
16. The anti-CD38 antibody for use according to any one of embodiments 12-15, wherein the anti-CD38 antibody is administered in combination with a second therapeutic agent.
17. The anti-CD38 antibody for use according to embodiment 16, wherein the second therapeutic agent is a proteasome inhibitor, cyclophosphamide or a corticosteroid.
18. The anti-CD38 antibody for use according to any one of embodiments 16-17, wherein the proteasome inhibitor is Velcade® (bortezomib) or NINLARO® (ixazomib).
19. The anti-CD38 antibody for use according to any one of embodiments 16-18, wherein the corticosteroid is dexamethasone.
20. The anti-CD38 antibody for use according to any one of embodiments 16-19, wherein the second therapeutic agent is Velcade® (bortezomib), NINLARO® (ixazomib), Kyprolis® (carfilzomib), Farydak® (panobinostat), cyclophosphamide, Alkeran® (melphalan), Thalomid® (thalidomide), Revlimid® (lenalidomide), Pomalyst® (pomalidomide), dexamethasone or interferon alpha.
21. The anti-CD38 antibody for use according to any one of embodiments 12-20, wherein the anti-CD38 antibody is administered in combination with a proteasome inhibitor, cyclophosphamide and a corticosteroid.
22. The anti-CD38 antibody for use according to embodiment 21, wherein the proteasome inhibitor is Velcade® (bortezomib).
23. The anti-CD38 antibody for use according to any one of embodiments 21-22, wherein the proteasome inhibitor is NINLARO® (ixazomib).
24. The anti-CD38 antibody for use according to any one of embodiments 21-23, wherein the corticosteroid is dexamethasone.
25. The anti-CD38 antibody for use according to any one of embodiments 21-24, wherein the proteasome inhibitor is Velcade® (bortezomib) and the corticosteroid is dexamethasone.
26. The anti-CD38 antibody for use according to any one of embodiments 21-25, wherein the proteasome inhibitor is NINLARO® (ixazomib) and the corticosteroid is dexamethasone.
27. The anti-CD38 antibody for use according to any one of embodiments 16-26, wherein the anti-CD38 antibody and the second therapeutic agent are administered simultaneously, sequentially or separately.
28. The anti-CD38 antibody for use according to any one of embodiments 16-27, wherein the anti-CD38 antibody and the second therapeutic agent are administered simultaneously, sequentially or separately.
29. The anti-CD38 antibody for use according to any one of embodiments 21-28, wherein the anti-CD38 antibody, proteasome inhibitor, cyclophosphamide and the corticosteroid are administered simultaneously, sequentially or separately.
30. The anti-CD38 antibody for use according to any one of embodiments 12-29, wherein AL is cardiac stage I, cardiac stage II, cardiac stage III, relapsed or refractory.
31. The anti-CD38 antibody for use according to any one of embodiments 12-30, wherein the patient is undergoing hematopoietic stem cell transplantation (HSCT).
32. The anti-CD38 antibody for use according to any one of embodiments 12-31, wherein the HSCT is allogeneic, autologous or syngeneic.
33. The anti-CD38 antibody for use according to any one of embodiments 12-32, wherein the HSCT comprises transplantation of blood stem cells derived from bone marrow, blood or amniotic fluid.
34. The anti-CD38 antibody for use according to any one of embodiments 12-33, wherein the anti-CD38 antibody is administered prior to, during or after HSCT.
35. The anti-CD38 antibody for use according to any one of embodiments 12-34, wherein the patient has completed chemotherapy and/or radiation therapy prior to HSCT.
36. The anti-CD38 antibody for use according to any one of embodiments 12-35, wherein the patient is further treated with radiotherapy.
37. The anti-CD38 antibody for use according to any one of embodiments 12-36, wherein the anti-CD38 antibody does not mediate killing of CD34-positive hematopoietic progenitor cells by complement dependent cytotoxicity (CDC).
38. The anti-CD38 antibody for use according to any one of embodiments 12-37, wherein the anti-CD38 antibody induces killing of CD38 positive plasma cells by antibody-dependent cell-mediated cytotoxicity (ADCC), antibody-dependent cellular phagocytosis (ADCP), complement dependent cytotoxicity (CDC), apoptosis, or modulation of CD38 enzymatic activity.
39. The anti-CD38 antibody for use according to any one of embodiments 12-38, wherein the anti-CD38 antibody competes for binding to CD38 with an antibody comprising a heavy chain variable region (VH) of SEQ ID NO: 4 and a light chain variable region (VL) of SEQ ID NO: 5.
40. The anti-CD38 antibody for use according to any one of embodiments 12-39, wherein the anti-CD38 antibody binds at least to the region SKRNIQFSCKNIYR (SEQ ID NO: 2) and the region EKVQTLEAWVIHGG (SEQ ID NO: 3) of human CD38 (SEQ ID NO: 1).
41. The anti-CD38 antibody for use according to any one of embodiments 12-40, wherein the anti-CD38 antibody comprises a heavy chain complementarity determining region 1 (HCDR1), a HCDR2 and a HCDR3 amino acid sequences of SEQ ID NOs: 6, 7 and 8, respectively, and a light chain complementarity determining region 1 (LCDR1), a LCDR2 and a LCDR3 amino acid sequences of SEQ ID NOs: 9, 10 and 11, respectively.
42. The anti-CD38 antibody for use according to any one of embodiments 12-41, wherein the anti-CD38 antibody comprises the VH comprising an amino acid sequence that is 95%, 96%, 97%, 98%, 99% or 100% identical to that of SEQ ID NO: 4 and the VL comprising an amino acid sequence that is 95%, 96%, 97%, 98%, 99% or 100% identical to that of SEQ ID NO: 5.
43. The anti-CD38 antibody for use according to any one of embodiments 12-42, wherein the anti-CD38 antibody comprises the VH of SEQ ID NO: 4 and the VL of SEQ ID NO: 5.
44. The anti-CD38 antibody for use according to any one of embodiments 12-43, wherein the anti-CD38 antibody comprises a heavy chain comprising an amino acid sequence that is 95%, 96%, 97%, 98%, 99% or 100% identical to that of SEQ ID NO: 12 and a light chain comprising an amino acid sequence that is 95%, 96%, 97%, 98%, 99% or 100% identical to that of SEQ ID NO: 13.
45. The anti-CD38 antibody for use according to any one of embodiments 12-44, wherein the anti-CD38 antibody comprises the heavy chain of SEQ ID NO: 12 and the light chain of SEQ ID NO: 13.
46. The anti-CD38 antibody for use according to any one of embodiments 12-46, wherein the anti-CD38 antibody comprises the HCDR1, the HCDR2, the HCDR3, the LCDR1, the LCDR2 and the LCDR3 of:
   a. the VH of SEQ ID NO: 14 and the VL of SEQ ID NO: 15;
   b. the VH of SEQ ID NO: 16 and the VL of SEQ ID NO: 17;
   c. the VH of SEQ ID NO: 18 and the VL of SEQ ID NO: 19; or
   d. the VH of SEQ ID NO: 20 and the VL of SEQ ID NO: 21, wherein the HCDR1, the HCDR2, the HCDR3, the LCDR1, the LCDR2 and the LCDR3 are defined by Kabat, Chothia, or IMGT.
47. The anti-CD38 antibody for use according to embodiment 46, wherein the anti-CD38 antibody comprises
   e. the VH of SEQ ID NO: 14 and the VL of SEQ ID NO: 15;
   f. the VH of SEQ ID NO: 16 and the VL of SEQ ID NO: 17;
   g. the VH of SEQ ID NO: 18 and the VL of SEQ ID NO: 19; or
   h. the VH of SEQ ID NO: 20 and the VL of SEQ ID NO: 21.
48. The anti-CD38 antibody for use according to any one of embodiments 12-47, wherein the anti-CD38 antibody is humanized or human.
49. The anti-CD38 antibody for use according to any one of embodiments 12-48, wherein the anti-CD38 antibody is of IgG1, IgG2, IgG3 or IgG4 isotype.
50. The anti-CD38 antibody for use according to any one of embodiments 12-49, wherein the anti-CD38 antibody is of IgG1 isotype.
51. The anti-CD38 antibody for use according to any one of embodiments 12-50, wherein the anti-CD38 antibody is administered intravenously.
52. The anti-CD38 antibody for use according to any one of embodiments 12-51, wherein the anti-CD38 antibody is administered subcutaneously in a pharmaceutical composition comprising the anti-CD38 antibody and a hyaluronidase.
53. The anti-CD38 antibody for use according to embodiment 42, wherein the hyaluronidase is rHuPH20 of SEQ ID NO: 22.

### Example 1. Materials and Methods

Mobilized blood stem and progenitor cells from MM and AL patients undergoing blood stem cell mobilization and collection on an IRB approved clinical study (Tufts Medical Center IRB #10680 Pre-Clinical Studies of DARZALEX™ (daratumumab) in Stem-Cell Mobilization and Transplant for Patients with Clonal Plasma cell Diseases) requiring written informed consent were used to study the effects of DARZALEX™ (daratumumab) on CD34 cell growth in vitro. Both unselected and CD34-selected mobilized blood progenitor cells from patients with MM and AL were used. The effects of DARZALEX™ (daratumumab) or an isotype control antibody was assessed on in vitro progenitor cell colony growth in semisolid assays as an indicator of DARZALEX™ (daratumumab)'s impact on the proliferation of CD34⁺ human hematopoietic progenitor cells.

Methylcellulose progenitor cell assays containing recombinant cytokines (Stem Cell Technologies, Vancouver, CA; Cat #04435) were performed according to manufacturer's instructions with unselected and CD34-selected mobilized blood stem cells from the day 1 leukapheresis product at several concentrations. CD34 selection was performed with the Miltenyi MiniMacs device. CD34-unselected cells were used at a concentration of 0.5×10⁴/ml and CD34-selected cells at 500 cells/ml. Assays were performed with DARZALEX™ (daratumumab) or control antibody at varying concentrations; in some assays, cells were plated in media containing DARZALEX™ (daratumumab) or control antibody, while in other assays cells were plated after incubating at 37° C. in 5% CO₂ for one hour in complement-rich human serum with DARZALEX™ (daratumumab) or control antibody. Colonies were counted on day 14 (CFU-GM, BFU-E, CFU-Mix).

### Example 2. CD38 is Expressed on Clonal AL Plasma Cells

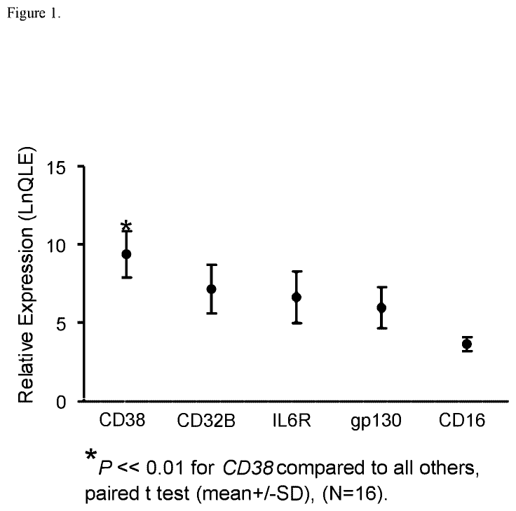

The clonal plasma cells of patients with AL express high levels of mRNA for CD38 based on transcriptional profiles of FACS-sorted CD138 bone marrow plasma cells (n=16, GEO GSE24128; Zhou et al., Clin Lymphoma Myeloma Leuk 12:49-58, 2012) obtained at diagnosis prior to therapy (FIG. 1). In addition, in an analysis of the immunophenotype of the CD138 bone marrow plasma cells from newly diagnosed AL patients, CD38 was uniformly found on the cell surface in all cases (Paiva et al., Blood 117:3613-6, 2011).

### Example 3. NK Cells from Patients with AL are Functional and Induce DARZALEX™ (Daratumumab)-Mediated ADCC Against CD38-Expressing Cells Three Weeks after Stem Cell Transplant (SCT)

With an IRB approved investigational protocol described in Example 1, requiring written informed consent, NK cells (CD3−/CD56+/CD16+) obtained three weeks post-SCT from AL patients were evaluated, assessing the percentage and number of NK cells in the peripheral blood specimens and the in vitro activity of these NK cell effectors with DARZALEX™ (daratumumab) in ADCC assays with human plasma cells (MM1S cells) as targets. MM1S cells express high levels of CD38, making them suitable targets for DARA. Flow cytometry was performed employing the FlowCellect™ Human Natural Killer Cell Characterization Kit (Millipore, Billerica, MA; catalogue #FCIM025164). The bioluminescent cytotoxicity assays (Cell Technology; Mountain View, CA) were performed following manufacturer's instructions. Target cell concentration was optimizedat 5000 MM1S cells per well. Target cells were incubated with DARZALEX™ (daratumumab) or control antibody (human IgG1 kappa, Sigma-Aldrich, St Louis MO) at 100 ng/mL for 15 minutes in a 37° C. 5% CO₂ incubator. Then, they were added to wells containing patient NK cells at 10:1 effector:target cell concentration. The number of NK cells in each sample was calculated based upon the flow cytometry characterization of the fresh patient specimens. The ADCC was determined by calculating luminescent values for each reaction condition as per manufacturer's instructions using standard controls. The amount of lysis per situation (% ADCC) was determined by the following calculation:

$$\% ADCC = ( Sample ) - ( Control 1 ) - ( Control 2 ) \times 100 ( Control 3 ) - ( Control 1 )$$

Sample: sample with DARZALEX™ (daratumumab)
Control 1: assay with isotype control (Target spontaneous release)
Control 2: no antibody, no effector cells
Control 3: maximum lysis in the presence of lysis reagent

The amount of DARZALEX™ (daratumumab) specific lysis was calculated by subtracting the % ADCC with isotype control from the % ADCC with DARZALEX™ (daratumumab).

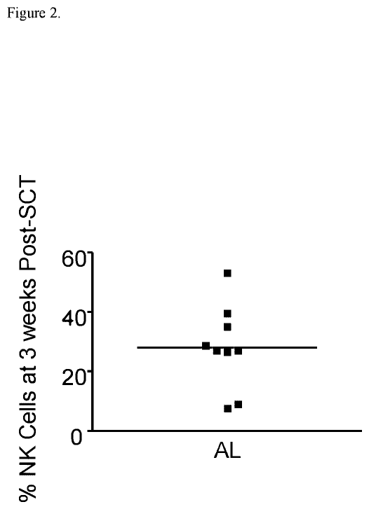

As shown in FIG. 2, NK cells were almost one third of all mononuclear cells in the peripheral blood of AL patients (n=9) at 3 weeks after SCT. In the bioluminescent ADCC assays, NK cells from patients with AL (n=6) combined with DARZALEX™ (daratumumab) had a median specific lysis of 32% (range, −3 to 73%).

### Example 4. DARZALEX™ (Daratumumab) does not Induce CD34+ Hematopoietic Progenitor Cell Killing Even Though High CD38 Expression is Found on these Cells

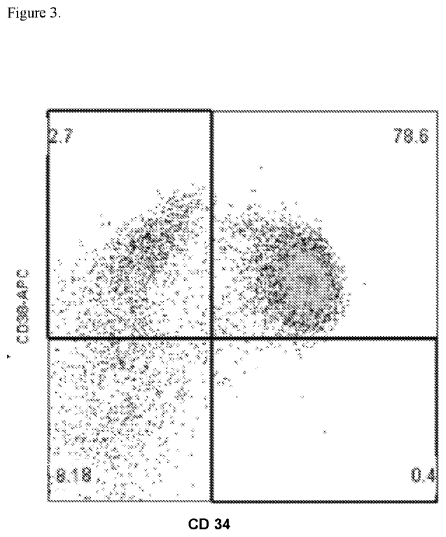

CD38 expression on CD34⁺ cells was analyzed using flow cytometry using APC-conjugated anti-human CD38 (HIT2, Biolegend, San Diego CA). The CD34⁺ cells had the appearance of myeloblasts, and expressed high levels of CD38 (FIG. 3).

The effects of DARZALEX™ (daratumumab) on CD34⁺ cell growth in vitro was studied using mobilized blood stem and progenitor cells from MM and AL patients undergoing blood stem cell mobilization and collection on an IRB approved clinical study requiring informed consent as described in Example 1. Both unselected and CD34-selected mobilized blood progenitor cells from patients with MM and AL were used, and the effects of daratumumab or an isotype control antibody on in vitro progenitor cell colony growth in semisolid assays was assessed as an indicator of DARZALEX™ (daratumumab)'s impact on the proliferation of CD34⁺ human hematopoietic progenitor cells.

Methylcellulose progenitor cell assays containing recombinant cytokines (Stem Cell Technologies, Vancouver, CA; Cat #04435) were performed according to manufacturer's instructions with unselected or CD34-selected mobilized blood stem cells from the day 1 leukapheresis product at several concentrations. CD34 selection was performed with the Miltenyi MiniMacs device. CD34-unselected cells were used at a concentration of 0.5×10⁴/ml and CD34-selected cells at 500 cells/ml. Assays were performed with DARZALEX™ (daratumumab) or control antibody at varying concentrations; in some assays, cells were plated in media containing DARZALEX™ (daratumumab) or control antibody, while in other assays cells were plated after incubating at 37° C. in 5% CO₂ for one hour in complement-rich human serum with DARZALEX™ (daratumumab) or control antibody. Colonies were counted on day 14 (CFU-GM, BFU-E, CFU-Mix).

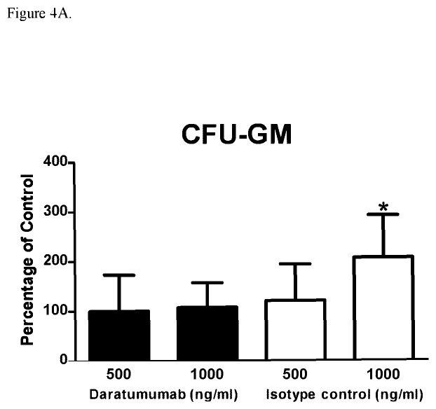

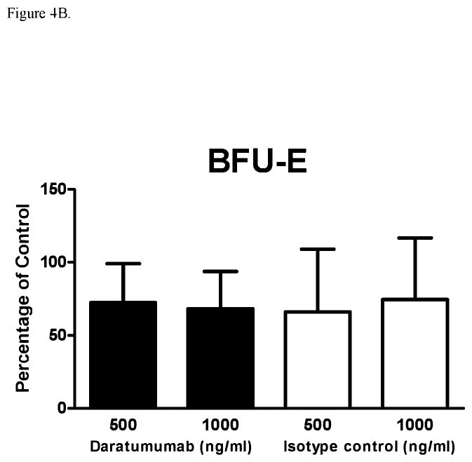

Thawed cryopreserved unselected mobilized blood progenitor cells grew similar numbers of CFU-GM (FIG. 4A) and BFU-E (FIG. 4B) in cultures with DARZALEX™ (daratumumab) or isotype control (500 or 1000 ng/ml, compared to no antibody control. In these experiments, thawed unselected mobilized human blood progenitor cells were plated at a concentration of 5×10⁴ cells per ml of semisolid medium (methylcellulose) in the presence of DARZALEX™ (daratumumab) or isotype control at 500 ng/ml or 1000 ng/ml. CFU-GM and GFU-E were counted two weeks later as percentage CFU-GM when compared to no antibody control. Of note, in the plates with 1000 ng/ml of isotype control antibody there was significantly more CFU-GM for unclear reasons.

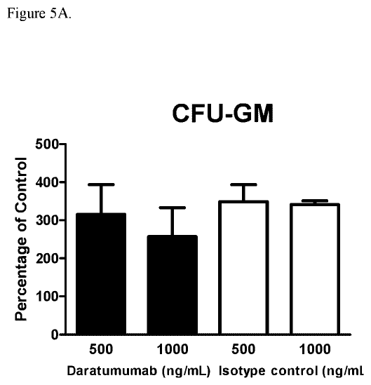

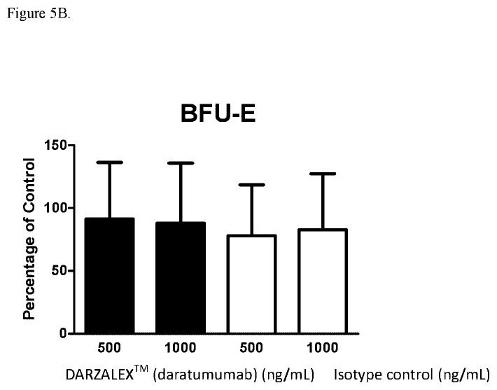

Effect of DARZALEX™ (daratumumab) on fresh unselected mobilized blood progenitor cells was also assessed. DARZALEX™ (daratumumab) did not reduce CFU-GM (FIG. 5A) or BFU-E (FIG. 5B). Both DARZALEX™ (daratumumab) and the isotype control had increased CFU-GM formation at similar levels when compared to the no antibody control.

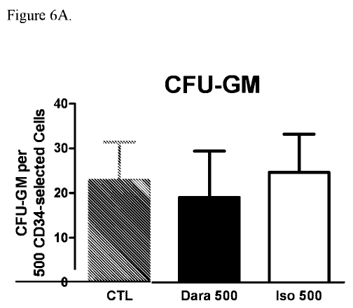

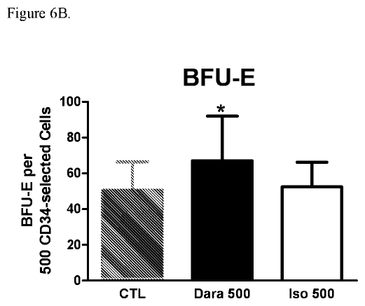

Ability of DARZALEX™ (daratumumab) to induce CDC on fresh CD34-selected hematopoietic progenitor cells was assessed. The CD34⁺ cells were incubated in 10% complement-rich human serum with no antibody, 500 ng/ml DARZALEX™ (daratumumab) or 500 ng/ml isotype control for 1 hour and then plated directly in semisolid medium. Colony formation was assessed per 500 CD34-selected cells. DARZALEX™ (daratumumab) did not reduce either CFU-GM (FIG. 6A) or BFU-E (FIG. 6B), indicating that the antibody did not induce CDC on the initial CD34-selected progenitor cells, despite the expression of CD38 on these cells. For unclear reason, there were more BFU-E in DARZALEX™ (daratumumab) treated samples. DARZALEX™ (daratumumab) increased the number of BFU-E formed.

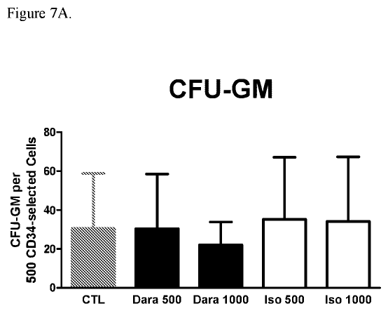

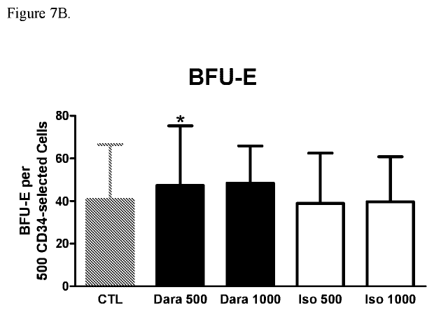

DARZALEX™ (daratumumab)'s effect on fresh CD34-selected hematopoietic progenitor cells was also tested by plating the cells directly into methylcellulose containing 500 ng/ml or 1000 ng/mL DARZALEX™ (daratumumab) or isotype control, or no antibody. Colony formation was assessed at day 14 for CFU-GM per 500 CD34-selected cells (FIG. 7A) or BFU-E per 500 CD34-selected cells (FIG. 7B). There was no decrease in colony formation with any concentration of DARZALEX™ (daratumumab), indicating that DARZALEX™ (daratumumab) was not toxic to CD34⁺ cell colony formation in this assay system. The results in FIGS. 6A, 6B and 7A show results from the mobilized blood progenitor cells of 1 patient with MM and 2 patients with AL. The results in FIG. 7B show results from the mobilized blood progenitor cells of 2 patients with MM and one patient with AL. Assays for BFU-E in the presence of 500 ng/ml DARZALEX™ (daratumumab) for unclear reasons contained significantly more BFU-E.

### Example 5. DARZALEX™ (Daratumumab)-Mediated ADCC by Patient NK Cells is Dependent on FcγRIIIA Genotype

Genomic DNA from patient cells was employed in PCR-based analysis using previously described methods and primers (Hatjiharissi et al., Blood 110:2561-2564, 2007) Amplicons were sequenced and analyzed for FcγRIIIA-158 polymorphisms. DARZALEX™ (daratumumab)-specific lysis in ADCC assays of patients with FcγRIIIA-158aa alleles encoding V/F and V/V was compared to F/F homozygotes.

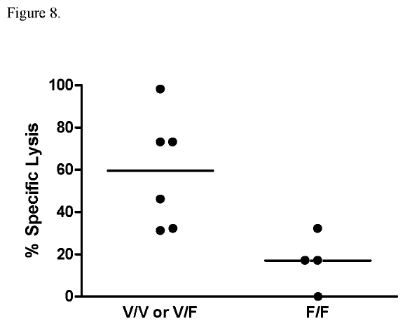

The PCR based-analysis showed that from ten patients analyzed in this study, six patients had V/F or V/V polymorphism and had a median 60% lytic activity (31-98) while 4 had F/F alleles and a median 17% lysis (0-32) (FIG. 8; P<0.05, Mann Whitney, two-tailed).

The initial correlation of Fc-receptor polymorphisms and DARZALEX™ (daratumumab) ADCC may be examined in future clinical trials.

### Example 6. a Randomized Phase 3 Study to Evaluate the Efficacy and Safety of DARZALEX™ (Daratumumab) in Combination with Cyclophosphamide, Bortezomib and Dexamethasone (CyBorD) Compared to CyBorD Alone in Newly Diagnosed Systemic AL Amyloidosis

A Phase 3, two-cohort, open-label study, comparing DARZALEX™ (daratumumab) in combination with CyBorD (Cyclophosphamide, Bortezomib, and Dexamethasone) to CyBorD alone in subjects with newly diagnosed AL Amyloidosis is conducted.

There are no currently approved medications for AL amyloidosis. In the absence of an approved treatment, medications developed for multiple myeloma are prescribed for the treatment. Combination of CyBorD is the most frequently used initial treatment for AL amyloidosis across EU and US (Venner et al., Blood 119:4387-4390, 2012; Mikhael et al., Blood 119:4391-4394, 2012; Jaggard et al., Hematologica 99:1479-1485, 2014; Palladini et al., Blood 126:612-615, 2015).

### Primary Objective

The primary objective is to evaluate the complete hematologic response following treatment with DARZALEX™ (daratumumab) in combination with CyBorD compared to CyBorD alone in AL patients.

### Secondary Objectives

To evaluate PFS based on all-cause mortality and progression disease (PD, including hematologic PD and organ PD according to consensus guidelines)

To evaluate the organ response rate (OrRR) (Comenzo 2012)
Kidney
Heart
Liver

To evaluate hematologic response rate (ORR) and hematologic VGPR or better (i.e., CR+VGPR) rate.

To evaluate organ progression rate for the heart, kidneys, liver.

To evaluate duration of and time to hematologic CR and VGPR or better, respectively.

To evaluate duration of and time to organ response

To assess the safety and tolerability of DARZALEX™ (daratumumab) when administered in combination with CyBorD.

To assess DARZALEX™ (daratumumab) pharmacokinetics

To assess the immunogenicity of DARZALEX™ (daratumumab)

To evaluate treatment effects on patient-reported outcomes (PROs) including the SF-36 Health questionnaire, EuroQol-5 Dimensions (EQ-5D-5L) and European Organization for Research and Treatment of Cancer (EORTC) QLQ-C30.

### Exploratory Objectives

To evaluate biomarkers of response including High Sensitivity (HS) Troponin T

To explore biomarkers predictive of response or resistance to therapy

To explore minimal residual disease status in amyloidosis patients

### Endpoints

### Primary Endpoint

The primary endpoint is complete hematological response rate.

### Secondary Endpoints

The secondary efficacy endpoints include:

Progression-free survival (PFS) based on all-cause mortality

Organ response rate (OrRR) for kidney, heart, liver

Overall survival (OS)

Time to next treatment (TNT)

Time to disease progression (TTP)

Time to hematologic disease progression

Overall Hematologic response

Hematologic VGPR or better rate

Time to complete hematologic response (or VGPR or better)

Duration of complete hematologic response (or VGPR or better)

Time to organ response

Duration of organ response

To evaluate treatment effects on patient reported outcomes including the EORTC QLQ-C30, Short Form-36 Health Survey [SF-36] and European Quality of Life Five Dimensions Questionnaire [EQ-5D-5L]).

### Exploratory Endpoint

The exploratory endpoint is to evaluate minimal residual disease status in patients who achieve complete hematologic response in bone marrow and blood

### Hypothesis

The primary hypothesis of this study is that is that DARZALEX™ (daratumumab) in combination with CyBorD will improve the complete hematological response rate compared to CyBorD alone, in subjects with newly diagnosed AL amyloidosis.

### Study Design:

This is a multicenter, Phase 3, two-cohort, open-label study, comparing DARZALEX™ (daratumumab) in combination with CyBorD to CyBorD alone in subjects with newly diagnosed AL. Approximately 360 subjects are randomized in two cohorts to initially receive either CyBorD or CyBorD in combination with DARZALEX™ (daratumumab) stratified by cardiac risk (Stage I, II, and IIIa). Each cycle is 4 weeks. DARZALEX™ (daratumumab) is administered at 16 mg/kg weekly for the first 2 cycles (8 weeks) of treatment, followed by every 2 weeks for 4 cycles (16 weeks) and then every 4 weeks until a maximum of 6 cycles of therapy (24 weeks) with the CyBorD backbone (both cohorts). Subjects randomized to the DARZALEX™ (daratumumab) group may continue DARZALEX™ (daratumumab) every 4 weeks after 6 cycles until disease progression for a maximum of 2 years. Subjects receive weekly cyclophosphamide 300 mg/m² either po or IV, bortezomib 1.3 mg/m² SQ, and dexamethasone 40 mg per week. Cycles are repeated every 4 weeks. A maximum number of 6 cycles are given.

Prior to randomization, a safety run-in is conducted in 6 subjects treated with DARZALEX™ (daratumumab) plus CyBorD for at least 1 cycle to establish safety of the combination regimen. Dosing of these 6 subjects is staggered such that subjects receive the first dose no sooner than 48 hours apart from the previously enrolled subject. Safety evaluation is performed by the sponsor and external experts after at least 1 cycle is completed for 6 subjects prior to the start of the randomized portion of the protocol. Subjects in the safety run-in continue all scheduled assessments as specified in the T&E, and contribute to the overall safety evaluation of DARZALEX™ (daratumumab)+CyBorD regimen. However, these subjects are not included in the overall efficacy assessment.

### Subject Selection

### Inclusion Criteria

1. Subject must be at least 18 years of age.
2. Histopathological diagnosis of amyloidosis or light chain deposition disease based on detection by polarizing microscopy of green bi-refringent material in Congo Red stained tissue specimens or characteristic electron microscopy appearance
3. Measurable disease of amyloid light chain amyloidosis as defined by at least ONE of the following: serum monoclonal protein >=0.5 g/dL by protein electrophoresis, >200 mg of monoclonal protein in the urine on 24 hour electrophoresis, serum free light-chain >=5.0 mg/dL with an abnormal kappa:lambda ratio or the difference between involved and uninvolved free light chains (dFLC) ≥5 mg/dL
4. Patient must have newly diagnosed AL Amyloidosis without prior systemic therapy. The only exception is that subjects may have up to 4 weeks of therapy with bortezomib, cyclophosphamide and/or dexamethasone (or equivalent steroid) prior to randomization on an emergency basis if the subject requires urgent therapy.
5. Eastern Cooperative Oncology Group (ECOG) Performance Status (PS) 0, 1 or 2
6. Subject must have pre-treatment clinical laboratory values meeting the following criteria during the Screening Phase:
   i) Absolute neutrophil count ≥1.0×10⁹/L;
   ii) Hemoglobin level ≥7.5 g/dL (≥5 mmol/L); (Blood transfusions to maintain Hb>7.5 is acceptable)
   iii) Platelet count >50×10⁹/L; Platelet transfusions are acceptable
   iv) Alanine aminotransferase level (ALT)≤2.5 times the upper limit of normal (ULN);
   v) Aspartate Aminotransferase (AST)≤2.5 times the upper limit of normal (ULN)
   vi) Total bilirubin level ≤1.5×ULN, (except for Gilbert Syndrome: direct bilirubin <2×ULN);
   vii) Creatinine clearance ≥=20 mL/min; Please note that the creatinine clearance can be either measured by 24 hour Urine study, or estimated using a validated equation, such as a the MDRD, CKD-epi, or Cockcroft Gault (see attachment 3 for details)
   viii) TSH and free T4 within normal limits. Patients may receive thyroid hormone therapy if needed to correct underlying hypothyroidism
7. Women of childbearing potential must be practicing a highly effective method of birth control 4 weeks prior to initiating treatment, during therapy, during the dose interruptions, and continuing for 4 weeks following discontinuation of study drugs. The birth control is consistent with local regulations regarding the use of birth control methods for subjects participating in clinical studies: e.g., established use of oral, injected or implanted hormonal methods of contraception; placement of an intrauterine device or intrauterine system; barrier methods: condom with spermicidal foam/gel/film/cream/suppository or occlusive cap (diaphragm or cervical/vault caps) with spermicidal foam/gel/film/cream/suppository (if hormonal or IUD contraception is medically contraindicated, 2 or other effective or highly effective methods may be used); male partner sterilization (the vasectomized partner should be the sole partner for that subject); true abstinence (when this is in line with the preferred and usual lifestyle of the subject) during and after the study (3 months after the last dose of DARZALEX™ (daratumumab) for women).
8. A man who is sexually active with a woman of childbearing potential and has not had a vasectomy must agree to use a barrier method of birth control, e.g. either condom with spermicidal foam/gel/film/cream/suppository or partner with occlusive cap (diaphragm or cervical/vault caps) with spermicidal foam/gel/film/cream/suppository. All men must also not donate sperm during the study and for 3 months after receiving the last dose of study drug.
9. A woman of childbearing potential must have 2 negative serum or urine pregnancy tests at Screening, first within 10 to 14 days prior to dosing and the second within 24 hours prior to dosing.
10. Each subject must sign an informed consent form (ICF) indicating that he or she understands the purpose of and procedures required for the study and are willing to participate in the study. Subjects must be willing and able to adhere to the prohibitions and restrictions specified in this protocol, as referenced in the ICF.

### Exclusion Criteria

1. Prior therapy for AL Amyloidosis or Multiple myeloma with the exception of a single cycle (maximum 4 weeks) of bortezomib, cyclophosphamide and/or dexamethasone (or equivalent steroid) prior to randomization
2. Previous or current diagnosis of symptomatic multiple myeloma defined by CRAB criteria, including the presence of lytic bone disease, plasmacytomas, and/or hypercalcemia.
3. Evidence of significant cardiovascular conditions as specified below:
   a) NT-ProBNP >8500 ng/L
   b) New York Heart Association (NYHA) classification IIIB or IV heart failure
   c) Unstable angina or myocardial infarction within 6 months prior to first dose
   d) Grade 2 or 3 atrioventricular (AV) block or sick sinus syndrome, unless subject has a pacemaker (Mobitz type I AV block of any grade is permitted)
   e) Known history of sustained (>30 second) ventricular tachycardia or cardiac syncope. Known history of recurrent non-sustained ventricular tachycardia (>3 beats) despite anti-arrhythmic therapy
   f) Screening 12-lead ECG showing a baseline QT interval as corrected (QTcF)>470 msec.
   g) Supine systolic blood pressure <90 mm Hg, or symptomatic orthostatic hypotension, or a decrease in systolic blood pressure upon standing of >20 mm Hg despite medical management (e.g. midodrine, fludrocortisones)
   h) Left Ventricular Ejection Fraction (LVEF) by transthoracic echocardiogram, MUGA scan, cardiac MRI or cardiac catheterization <40%. Assessment required during screening.
4. Subjects planning to undergo a stem cell transplant during first six cycles of protocol therapy are excluded. Stem cell collection during the first six cycles of protocol therapy is permitted.
5. Diagnosed or treated for malignancy other than AL, except:
   a) Malignancy treated with curative intent and with no known active disease present for ≥5 years before randomization
   b) Adequately treated non-melanoma skin cancer or lentigo maligna without evidence of disease
   c) Adequately treated carcinoma in situ (e.g. cervical, breast) with no evidence of disease
6. Subject has known chronic obstructive pulmonary disease (COPD) with a Forced Expiratory Volume in 1 second (FEV1)<50% of predicted normal. Note that FEV1 testing is required for patients suspected of having COPD and subjects must be excluded if FEV1<50% of predicted normal.
7. Subject has known moderate or severe persistent asthma within the past 2 years (see Attachment 5), or currently has uncontrolled asthma of any classification. (Note that subjects who currently have controlled intermittent asthma or controlled mild persistent asthma are allowed in the study).
8. Subject is known to be seropositive for human immunodeficiency virus (HIV), known to have hepatitis B surface antigen positivity, or known to have a history of hepatitis C.
9. Grade 3 sensory or grade 1 painful peripheral neuropathy
10. Known hypersensitivity to bortezomib, boron or mannitol
11. Subject has any concurrent medical condition or disease (e.g., active systemic infection) that is likely to interfere with study procedures or results, or that in the opinion of the investigator would constitute a hazard for participating in this study.
12. Any form of secondary or familial (ATTR) amyloidosis
13. Subject has known allergies, hypersensitivity, or intolerance to monoclonal antibodies or human proteins, or their excipients (refer to Investigator Brochure), or known sensitivity to mammalian-derived products.
14. Subject is known or suspected of not being able to comply with the study protocol (e.g., because of alcoholism, drug dependency, or psychological disorder) or the subject has any condition for which, in the opinion of the investigator, participation would not be in the best interest of the subject (e.g., compromise their well-being) or that could prevent, limit, or confound the protocol-specified assessments.
15. Subject is a woman who is pregnant or breast-feeding or planning to become pregnant while enrolled in this study or within 6 months after the last dose of study drug.
16. Subject has received an investigational drug (including investigational vaccines) or used an invasive investigational medical device within 4 weeks before Cycle 1, Day 1 (except for investigational anti-myeloma agents, which cannot be taken within 2 weeks prior to Cycle 1, Day 1, as described in exclusion #3).
17. Subject has had major surgery within 2 weeks before Cycle 1, Day 1, or will not have fully recovered from surgery, or has surgery planned during the time the subject is expected to participate in the study or within 2 weeks after the last dose of study drug administration. Note: subjects with planned surgical procedures to be conducted under local anesthesia may participate.

### Safety Evaluations

Safety will be measured by adverse events, laboratory test results, ECGs, vital sign measurements, physical examination findings, and ECOG performance status. Any clinically relevant changes occurring during the study will be recorded on the Adverse Event section of the eCRF. Any clinically significant abnormalities persisting at the end of the study/early withdrawal will be followed by the investigator until resolution or until a clinically stable endpoint is reached.

### Efficacy

### Response Categories

Disease evaluations will be performed every 28 days on the scheduled assessment day (±3 days). If treatment has been delayed for any reason, then the disease evaluations will be performed according to schedule, regardless of any changes to the dosing regimen.

Disease evaluations will be performed by a central laboratory (unless otherwise specified) according to the Time and Events Schedules until disease progression. This study will use the consensus recommendations for AL amyloidosis treatment response criteria (Comenzo et al., Leukema 26:2317-2325, 2012) presented below. For free light chain assessment, quantitative immunoglobulin, M-protein, and immunofixation measurements in serum and 24 hour urine, the investigator will use results provided by the central laboratory. Subjects with positive serum IFE and confirmed DARZALEX™ (daratumumab) IFE interference, that meet all other clinical criteria for complete response, will be considered CR.

### International Uniform Response Criteria Consensus Recommendations

Hematologic response and progression criteria

| Response | |
|---|---|
| Category | Criteria |
| Complete | Normalization of free light chain levels and |
| | ratio, negative serum and urine immunofixation |
| Very Good Partial | Reduction in the dFLC < 40 mg/L |
| Partial | A greater than 50% reduction in the dFLC |
| No response | Less than a PR |
| Progression | From CR, any detectable monoclonal protein |
| | or abnormal free light chain ratio (light chain |
| | must be double) |
| | From PR, 50% increase in serum M-protein to |
| | >0.5 g/dl or 50% increase in urine |
| | M-protein to >200 mg/day (a visible peak must |
| | be present) |
| | Free light chain increase of 50% to >100 mg/L |

Abbreviations:
CR, Complete response;
dFLC difference between iFLC and uninvolved FLC;
FLC free light chain;
PR, partial response

### Example 7. a Randomized Phase 2 Study to Evaluate the Efficacy and Safety of DARZALEX™ (Daratumumab) as a Single Agent for the Treatment of Subjects with Systemic AL Amyloidosis

A Phase 2 open-label study evaluating safety and efficacy of DARZALEX™ (daratumumab) as a single agent in subjects with systemic AL amyloidosis who have received treatment previously is conducted.

Approximately 40 subjects are randomized in two cohorts, one receiving DARZALEX™ (daratumumab) and the other one receiving placebo.

Adult patients 18 years or older with biopsy-proven systemic AL amyloidosis who are not in CR or VGPR after initial treatment, Mayo Clinic Cardiac stage I and II patients are included, stage III patients are included only with a NT-proBNP≤5000 ng/l (or BNP≤1000 ng/l).

### Dosing Regimen

Two dosing regimens are considered.

### Regimen 1:

DARZALEX™ (daratumumab) is dosed 16 mg/kg IV weekly×8 doses, every other week×8 doses and then every 4 weeks until 6 cycles. After 6 cycles patients will continue DARZALEX™ (daratumumab) every 4 weeks until progression or elective discontinuation

### Regimen 2:

Daratumumab is dosed for six 28-day cycles, 16 mg/kg administered by IV route.

For the first cycle, DARZALEX™ (daratumumab) is administered weekly at days 1, 8, 15, and 22

For cycles 2 and 3, DARZALEX™ (daratumumab) is administered every other week at days 1 and 15

From cycle 4 through 6, DARZALEX™ (daratumumab) is administered every 4 weeks at day 1

Primary objective, secondary objectives, key inclusion criteria and key exclusion criteria are similar to those described in Example 6.

What is claimed is:

1. A method of treating light chain amyloidosis (AL) in a newly diagnosed AL patient, comprising subcutaneously administering daratumumab to the patient in combination with bortezomib, cyclophosphamide, and dexamethasone.

2. The method of claim 1, wherein the daratumumab is administered in a dosing regimen of sequential 4-week cycles, wherein 1,800 mg daratumumab is administered weekly for Cycles 1 and 2, every two weeks for Cycles 3 to 6, and every 4 weeks for Cycle 7 and subsequent cycles.

3. The method of claim 2, wherein:
a) about 1.3 mg/m² bortezomib;
b) about 300 mg/m² cyclophosphamide; and
c) about 20 mg of dexamethasone,
are administered on Days 1, 8, 15, and 22 of each of the sequential 4-week cycles of the dosing regimen.

4. The method of claim 2, wherein:
a) about 1.3 mg/m² bortezomib;
b) about 300 mg/m² cyclophosphamide; and
c) about 40 mg of dexamethasone,
are administered on Days 1, 8, 15, and 22 of each of the sequential 4-week cycles of the dosing regimen.

5. The method of claim 4, wherein administration of the dosing regimen to the patient continues until disease progression or for a maximum of two years.

6. The method of claim 4, wherein the patient is undergoing hematopoietic stem cell transplantation (HSCT).

7. The method of claim 6, wherein the HSCT is allogeneic.

8. The method of claim 6, wherein the HSCT is autologous.

9. The method of claim 6, wherein the HSCT is syngeneic.

10. The method of claim 6, wherein the daratumumab is administered prior to the HSCT.

11. The method of claim 6, wherein the daratumumab is administered during or after the HSCT.

12. The method of claim 6, wherein the patient does not have New York Heart Association (NYHA) classification IIIB or IV heart failure.
